(19) United States
(12) Patent Application Publication
(10) Pub. No.: US 2003/0215791 A1
(43) Pub. Date: Nov. 20, 2003

(54) Method of and system for multiplexed analysis by spectral imaging

(75) Inventors: Yuval Garini, Doar Na Misgav (IL); Nir Katzir, Givat Elah (IL); Irit Bar-Am, Herzlia (IL); Uri Milman, Migdal HaErnek (IL); Eli Horn, Kiryat Motzkin (IL); Yacov Malinovich, Tivon (IL); Terry Hammill, La Mesa, CA (US); Vladimir Cherepakhin, Oceanside, CA (US)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Applied Spectral Imaging Ltd.

(21) Appl. No.: 10/277,916

(22) Filed: Oct. 23, 2002

Related U.S. Application Data
(60) Provisional application No. 60/381,353, filed on May 20, 2002.

(51) Int. Cl. C12Q 1/70; C12Q 1/68; G01N 33/53; G01N 33/554; G01N 33/569; G06F 19/00; G01N 33/48; G01N 33/50

(52) U.S. Cl. 435/5; 435/6; 435/7.1; 435/7.32; 702/19; 702/20

(57) ABSTRACT

A method of detecting the presence, absence and/or level of a plurality of analytes-of-interest in a sample, the method comprisES: (a) providing a plurality of objects, each of the plurality of objects having a predetermined, measurable and different imagery characteristic, and further having a predetermined and specific affinity to one analyte of the plurality of analytes-of-interest, each the imagery characteristic corresponding to one the predetermined specific affinity, hence each the imagery characteristic corresponds to one analyte of the plurality of analytes-of interest; (b) providing at least one affinity moiety having a predetermined and specific affinity or predetermined and specific affinities to the plurality of analytes-of-interest, each the affinity moiety having a predetermined, measurable response to light; (c) combining the objects, the at least one affinity moiety and the sample under conditions for affinity binding; and (d) simultaneously determining, for each object of the plurality of objects an imagery characteristic, and for at least a portion of the at least one affinity moiety a response to light, thereby detecting the presence, absence and/or level of the plurality of analytes-of-interest in the sample.

### FIELD AND BACKGROUND OF THE INVENTION

The present invention relates to a method and system for the analysis of biological samples, and, more particularly, to a method and system for the simultaneous detection of the presence, absence and/or level of a plurality of analytes-of-interest that may be present in an analyzed sample.

Various procedures are commonly employed to determine the presence, absence, and/or level (e.g., amount, concentration) of substances of clinical or research significance which may be present in biological samples, such as biological fluids or extracts, including, but not limited to, urine, whole blood, plasma, serum, sweat, saliva, tears, wound secretions and other body fluids or homogenized or substantially intact tissues and/or cells. Such substances are commonly referred to as analytes, and are referred to herein as analytes-of-interest, which may include small to large compounds, ranging from hormones and fats, to bio-polymers such as proteins, nucleic acids and complex carbohydrates.

Affinity is one characteristic of molecules participating in binding as “binding pairs”, such as, for example, enzyme-substrate, enzyme-inhibitor, antibody-antigen, receptor-ligand and polynucleotide-complementary polynucleotide. As is well known in the art, affinity can be used to determine the presence, absence, and/or level of an analyte-of-interest which may be present in a biological samples, by quantitatively or qualitatively monitoring the binding of the analyte-of-interest to a counterpart member of a binding pair.

An antibody is a molecule produced by the immune system of animals, typically in response to the introduction of a foreign entity such as a pathogen. In this respect a foreign entity is also called an antigen. An antibody forms very strong bonds to a particular portion of a respective antigen, known as a hapten; a single antigen typically includes several different haptens, whereby any particular antibody binds to a single unique hapten. This recognition and subsequent binding are among the initial stages of an immune response.

Antibodies can be used for diagnostics procedures in various ways. The underlying principle of using antibodies in diagnostics is the ability to qualitatively or quantitatively determine the presence or measure the amount of antibody that reacted with a tested material.

Thus, for example, in some diagnostic procedures, labeled antibodies, specific for an analyte-of-interest, are applied to a strip of absorbent material through which labeled antibodies in solution can flow via capillarity. By immobilizing a test sample in a particular portion of the strip, i.e., capture zone, and measuring the amount of labeled antibody which is captured thereat through specific binding, the concentration of analyte in the test sample can be semi-quantitatively determined.

However, detection of multiple analytes or separately identifiable characteristics of one or more analytes, through single-step assay processes provide for very limited capabilities, in contradiction to the general tendency in developing and using highthroughput assays.

The capability of simultaneously performing multiple determinations through a single process is known as “multiplexing” and a process that implements such a capability is called a “multiplexed assay”. Novel highly multiplexed and highthroughput assays are currently sought for in many disciplines in the arts of biological research and medical diagnostics.

Naturally occurring nucleic acids, i.e., DNA and RNA, provide for the information required to synthesize proteins, which dictate and regulate structure and function (phenotype) at the subcellular, cellular and organism levels. Nucleic acids are often found double stranded, whereby the strands have high sequence dependent, binding affinity and specificity towards one another. The nature and distribution of various RNA molecules expressed in different cell types, e.g., pathological cells such as cancer cells, and different times in a given cell type can shed light on the functionality of the proteins involved in normal and pathological cellular processes. Similarly, the nature and distribution of various DNA sequences present in different species and different individuals of the same species can shed light on phylogenetic relations among organisms and evolution processes; and on the genetic make-up of given individuals.

As is further entailed below, there is an increasing need to have multiplexed highthroughput assays with which to screen nucleic acids for the purpose of directly (e.g., mRNA expression levels) or indirectly (e.g., SNPs linkage analysis) identifying sequence involved in a variety of pathologies.

Identification of sequences is of major importance in life science, which has progressed to the realization of the importance of the interaction of the genome and environmental factors in the etiology of the majority of the multifactorial, more complex, disorders. The more complete and reliable the correlation established between gene expression and health or disease states, the better diseases can be described, diagnosed and treated. The state of gene expression at any time in any given cell is represented by the composition of mRNA, which is synthesized by regulated transcription of the DNA in that cell. Consequently, rapid detection of mRNA expression levels in biological samples is desired.

Highthroughput technologies for gene expression analysis not only helps to better understand and characterize the diseased and healthy states, it may also assist in drug development, in determining the mechanistic basis for drug action and toxicity and in individualizing drug therapy.

A correlation between a response to a drug and genomic variability may also be established indirectly by analyzing single nucleotide polymorphisms (SNPs) which were in some cases shown to be predictive markers for such correlation. Estimations show that the fraction of SNPs is about 0.1% base pairs, and that the total amount of SNPs in the human genome is larger than 3 million. Thus, SNPs offer a potential for (i) identification of disease-causing genes and candidate drug targets; (ii) development and redefining of lo diagnostics; and (iii) establishment of markers for individualized medicine.

One commonly employed highthroughput screening method is by a microtiter plate carrying a plurality of samples, each confined in one location of the microtiter plates. Miniaturized high-density microtiter plates having densities of up to 3456-wells per one plate are commercially available [B. J. Battersby et al., “Novel Miniaturized Systems in High-Throughput Screening”, Trends in Biotech 20:167-173 (2002), the contents of which are hereby incorporated by reference]. Although the use of high density microtiter plates significantly increase the overall throughput screening, such methods are intrinsically limited by (i) the physical constraints of delivering small volumes to wells; (ii) the theoretical minimum number of molecules needed to interact to ensure binding; and (iii) the ability to rapidly and sensitively detect responses [L. Silverman et al., “New assay technologies for high-throughput screening”, Current Opinion in Chemical Biology 2:397-403 (1998)]. Thus, as the technological density limits are insufficient for high throughput screening, the number of microtiter plates screened per day is continuously increasing and the use of expensive robotic systems is unavoidable. This approach has a significant environmental impact due to the increased number of plates and reaction mixture solutions generated for post analysis disposal. In addition, storage space for the increased number of plates is also becoming an important consideration.

There is a recognized need to simultaneously conduct large numbers of assays within one well, thereby to push high throughput screening to the next level of screening capabilities.

In recent years chips carrying an array of affinity biomolecules, such as single strand DNA, oligonucleotides, antibodies, proteins were developed, whereby a single chip can carry thousands of different biomolecules. Nevertheless, there is a limitation to the density of different biomolecules placeable on a chip both at the production and detection level.

Over the past few decades, small particles, also known in the relevant literature as beads or microspheres, have become a powerful tool for determining the presence, absence and/or level of analytes-of-interest in a sample. Beads are used in numerous biochemical studies such as diagnostics, cell-separation, protein purification and the like. Columns with various beads are used for affinity, size exclusions and ionic strength separation and purification.

For example, beads are useful for isolation of rare cells from a heterogeneous cell population. The cell suspension is mixed with a specific antibody that has been conjugated to a small sized bead, which binds to specific markers unique to the rare cell. Subsequently the beads are collected as a homogeneous group by an outer manipulation, e.g., ultra centrifugation, filtration, magnet and the like.

For efficient use, the beads must be sufficiently small (typically in a micrometer scale) so that the suspension period (before sinking) of the beads would be long. In addition, the smallness of the beads provides a relatively large reactive surface area and increases the collisions rate of the beads with the target analyte in solution. In order to enable a bead to be used for the detection of analytes, a suitable affinity moiety, having affinity to the analyte, is applied to the bead. The affinity moiety may be adsorbed onto the surface of each bead or it can be bound, e g., by covalent linking, to a functionalized chemical group on the bead.

Beads are available with a variety of functional surfaces, densities, shapes and physical properties, e.g., magnetic and/or optical properties. In particular, colored or fluorescent beads have become an important feature for assay development, providing numerous benefits such as multiplexing and signal enhancement. Fluorescent beads serve as a replacement for radioactive labels [Meza, M. B. “Bead-based High Throughput Screening applications in drug discovery”, Drug. Disc. Today: HTS Supplement 2000, 1(1):38-41].

When a fluorochrome molecule (also referred to herein as a fluorophore molecule) embedded in a bead absorbs light, electrons are boosted to a higher energy shell of an unstable excited state. During the lifetime of excited state (typically 1-10 nanoseconds) the fluorochrome molecule undergoes conformational changes and is also subject to a multitude of possible interactions with its molecular environment. The energy of excited state is partially dissipated, yielding a relaxed singlet excited state from which the excited electrons fall back to their stable ground state, emitting light of a specific wavelength. The emission spectrum is shifted towards a longer wavelength than its absorption spectrum. The difference in wavelength between the apex of the absorption and emission spectra of a fluorochrome (also referred to as the Stokes shift), is typically small.

Not all the molecules initially excited by absorption return to the ground state by fluorescence emission. Other processes such as collisional quenching, fluorescence resonance energy transfer and intersystem crossing may also depopulate the excited state. A ratio of the number of fluorescence photons emitted to the number of photons absorbed, called “fluorescence quantum yield”, is a measure of the relative extent to which these processes occur. For fluorochromes which are commercially available, only a small portion (about 0.1%) of the absorbed light is actually emitted.

The low fluorescence quantum yield and the small separation between the absorption and emission spectra, require the usage of spectral discrimination methods to allow a clear detection. Typically, the discrimination methods utilize a set of filters on the excitation path and emission path of a fluorescence detection system. Such filters were greatly developed during the past years, and are being manufactured by various companies such as Chroma Technology (Brattleboro, Vt. USA) and Omega Optics (Brattleboro, Vt., USA).

Fluorophore beads are useful also in detection procedures known as flow cytometry. Flow cytometry is an optical technique that analyzes beads and other particles, e.g., cells, in a fluid mixture based on optical characteristics of the beads using a device known as a flow cytometer. Using hydrodynamic means, flow cytometers focus a fluid suspension of beads into a thin stream so that the particles flow down the stream in a substantially single file and pass through an examination zone. A focused light beam, such as a laser beam, illuminates the beads as they flow through the examination zone. Optical detectors within the flow cytometer measure certain characteristics of the light as it interacts with the beads.

To date, flow cytometry has been unsatisfactory as applied to provide a fully multiplexed assay capable of real-time analysis of more than a few different analytes. In addition, in flow cytometry the beads are detected one by one in the examination zone, using a single point detector (typically a photomultiplier or a photodiode). Hence, although in flow cytometry a plurality of bead characteristics may be used in a single measurement, the time of measurement is proportional to the number of beads and it may be, in principle, considerably large. On the other hand, if the flow rate of the beads is high, the measurement of each bead passing through the examination zone has to be performed within a small fraction of time, which is inversely proportional to the velocity of the beads. It would be appreciated by one ordinarily skilled in the art that small measurement time decreases the amount of information which can be collected from any given bead. For performing a precise, accurate and therefore reliable measurement employing flow cytometry, the flow rate should thus be sufficiently small.

Hence, an inherent drawback of flow cytometry is that multiplexing and information are two conflicting features; it is inevitable that increasing of one feature is accompanied by a decrement of the other.

In other prior art methods the sample of interest is placed in several small confined volumes, for the purpose of separately detecting the fluorescence intensity of each portion of the sample. One known such method is Enzyme Linked Immunosorbent Assay (ELISA), where the detection is carried out, for example, in a 96-wells microtiter plate. ELISA is advantageous since all the reactions can be carried out in the wells of the plate.

Nowadays, a variety of dedicated ELISA instruments are available, e.g., ELISA plate readers (modified spectrophotometers) and ELISA plate washers. Similarly to the flow cytometry method, the optical detection of the samples in the microtiter plate is based on a single point detector and the measurements are of a single fluorescence intensity value per well. Thus, although the biochemical reactions simultaneously occur in each of the confined volumes, the detection itself is linear in the number of confined volumes and in that sense the method cannot be considered as a multiplexed assay. Moreover, known ELISA systems have limited spatial and spectral resolutions which is insufficient for identifying each bead separately.

In number of assay methods a single ELISA procedure is replaced with flow cytometry. An example is the measurement of the DNA index, intensively used for tumors diagnostics. These methods, described for example in “Flow Cytometry, Practical Approach”, ed. M. G. Ormerod, IRL Press, Oxford University Press 1994 (see also www.partec.de) however, are based on only a few characteristics of the beads under analysis hence allow determination of limited number of analytes per assay. Moreover, due to software limitations, the analytic determinations in prior art methods hamper the overall procedure.

Additional prior art of relevance includes: U.S. Pat. Nos. 5,736,330, 5,981,180, 6,057,107, 6,139,800, 6,160,618, 6,268,222, 6,337,472 and 6,366,354.

The present invention provides solutions to the problems associated with prior art techniques aimed at multiplexed analysis of a plurality of analytes-of-interest.

### SUMMARY OF THE INVENTION

According to one aspect of the present invention there is provided a method of detecting the presence, absence and/or level of a plurality of analytes-of-interest in a sample, the method comprising: (a) providing a plurality of objects, each of the plurality of objects having a predetermined, measurable and different imagery characteristic, and further having a predetermined and specific affinity to one analyte of the plurality of analytes-of-interest, each the imagery characteristic corresponding to one predetermined specific affinity, hence each imagery characteristic corresponds to one analyte of the plurality of analytes-of interest; (b) providing at least one affinity moiety having a predetermined and specific affinity or predetermined and specific affinities to the plurality of analytes-of-interest, each affinity moiety having a predetermined, measurable response to light; (c) combining the objects, the at least one affinity moiety and the sample under conditions for affinity binding; and (d) simultaneously determining, for each object of the plurality of objects an imagery characteristic, and for at least a portion of the at least one affinity moiety a response to light, thereby detecting the presence, absence and/or level of the plurality of analytes-of-interest in the sample.

According to still further features in the described preferred embodiments the predetermined, measurable and different imagery characteristic is selected from the group consisting of a unique size, a unique geometrical shape and a unique response to light.

According to still further features in the described preferred embodiments the step (d) is by a spectral imaging device operable to construct a spectral image of the sample.

According to still further features in the described preferred embodiments the step (d) comprises determining, for each object, a wavelength value and an intensity value.

According to still further features in the described preferred embodiments the wavelength value is used to determine a presence of a particular analyte of the plurality of analytes-of-interest in the sample.

According to still further features in the described preferred embodiments the method further comprising repeating the step (c) a plurality of times, each time on a different x-y location of a two-dimensional platform.

According to still further features in the described preferred embodiments the step (d) is performed for each x-y location separately.

According to still further features in the described preferred embodiments the step (d) is performed simultaneously for all x-y locations.

According to still further features in the described preferred embodiments the method further comprising repeating the step (d) at least once, so as to optimize a signal-to-noise ratio.

According to still further features in the described preferred embodiments the method further comprising performing at least one calibration spectral imaging measurement prior to the step (d).

According to still further features in the described preferred embodiments the responses to light of the plurality of objects and the responses to light of the at least one moiety are determined simultaneously.

According to still further features in the described preferred embodiments responses to light of the plurality of objects and responses to light of the at least one moiety are determined separately and independently.

According to still further features in the described preferred embodiments responses to light of the at least one moiety are determined by gray-level imaging.

According to still further features in the described preferred embodiments the method further comprising subtracting background spectra from the spectral image, the background spectra are collected from a regions of the image which are characterized by absence of objects.

According to still further features in the described preferred embodiments the method further comprising magnifying the spectral image by a magnification factor, the magnification factor is from 1 to 100.

According to still further features in the described preferred embodiments the method further comprising selecting an optimal excitation and emission spectrum of each of the plurality of objects.

According to still further features in the described preferred embodiments the selecting an optimal excitation and emission spectrum is by an epi-fluorescent setup which comprises at least one spectral filter.

According to still further features in the described preferred embodiments the step (d) is effected by a procedure selected from a group consisting of a principle component analysis, a principle component regression and a spectral decomposition.

According to still further features in the described preferred embodiments the step (d) comprises using a library of reference spectra characterizing the plurality of objects.

According to still further features in the described preferred embodiments the step (d) comprises: (i) illuminating the sample with incident light; and (ii) collecting exiting light from the sample so as to acquire a spectrum of each object of the plurality of objects.

According to still further features in the described preferred embodiments the method further comprising positioning at least a portion of the plurality of objects on a two-dimensional platform, prior to the step (i).

According to still further features in the described preferred embodiments the positioning is effected by a procedure selected from the group consisting of printing and gluing.

According to still further features in the described preferred embodiments the method further comprising using at least one filter to adjust a spectrum of the incident light.

According to still further features in the described preferred embodiments the method further comprising substantially filtering out an exciting wavelength of the incident light while collecting the exiting light.

According to still further features in the described preferred embodiments the filtering out exciting wavelength is by an optical device selected from the group consisting of a dichroic mirror, a dark-field objective lens, a phase contrast device and a Numarski-prism.

According to still further features in the described preferred embodiments the method further comprising acquiring an intensity value of each picture element of the at least a portion of the sample.

According to still further features in the described preferred embodiments the intensity value is used to determine a level of a particular analyte of the plurality of analytes-of-interest in the sample.

According to still further features in the described preferred embodiments the step (ii) is characterized by spectral resolution ranging between 1 nm and 50 nm and spatial resolution ranging between 0.1 μm and 1.0 μm.

According to still further features in the described preferred embodiments the method further comprising generating individual spectra-images from spectra acquired in the step (ii).

According to still further features in the described preferred embodiments the illuminating is by at least one light source selected from the group consisting of Mercury lamp, Xenon lamp, Tungsten lamp, Halogen lamp, laser light source, Metal-Halide lamp.

According to still further features in the described preferred embodiments the spectral imaging device comprises a dispersion element and a detector.

According to still further features in the described preferred embodiments the dispersion element is an interferometer.

According to still further features in the described preferred embodiments the step (d) comprises: (i) collecting incident light simultaneously from the plurality of objects; (ii) passing the incident light through the interferometer, so that the light is first split into two coherent beams having an optical path difference therebetween, and then the two coherent beams recombine to interfere with each other to form an exiting light; (iii) focusing the exiting light on the detector, so that each of the detector elements produces a signal which is a particular linear combination of light intensity emitted by a respective object of the plurality of objects, the linear combination is a function of the optical path difference; (iv) simultaneously scanning the optical path difference for the plurality of objects; and (v) recording the signals of each of the detector elements as function of time.

According to still further features in the described preferred embodiments the method further comprising passing the incident light through a collimator, prior the step (ii), where the collimator designed and configured such that the light is simultaneously collected and collimated for each of the plurality of objects.

According to still further features in the described preferred embodiments the simultaneously scanning the optical path difference is by rigidly rotating the beam-splitter and the two mirrors around an axis perpendicular to a plane formed by the two coherent beams.

According to still further features in the described preferred embodiments the interferometer further comprises a first periscope mirror, a second periscope mirror and a double sided mirror having a first side and a second side, wherein the simultaneously scanning the optical path difference is by rotating the double sided mirror around an axis perpendicular to a plane formed by the two coherent beams, in a manner that the incident light: encounters the first side of the double sided mirror, encounters the first periscope mirror, splits and recombined in the beam-splitter and the two mirrors; encounters the second periscope mirror, and encounters the second side of the double sided mirror.

According to still further features in the described preferred embodiments the interferometer further comprises a single large mirror, wherein the simultaneously scanning the optical path difference is by rotating the large mirror around an axis perpendicular to a plane formed by the two coherent beams, in a manner that the incident light: encounters the large mirror; splits and recombined in the beam-splitter and the two mirrors; and reflected by the large mirror.

According to still further features in the described preferred embodiments the method further comprising simultaneously transferring all data in real time from all the elements of the detector array to a computer, and displaying an image on an output device.

According to another aspect of the present invention there is provided a system for detecting the presence, absence and/or level of a plurality of analytes-of-interest in a sample, the system comprising: (a) a plurality of objects, each of the plurality of objects having a predetermined, measurable and different imagery characteristic, and further having a predetermined and specific affinity to one analyte of the plurality of analytes-of-interest, each the predetermined imagery characteristic corresponding to one the predetermined specific affinity, hence each the imagery characteristic corresponds to one analyte of the plurality of analytes-of interest; (b) at least one affinity moiety having a predetermined and specific affinity or predetermined and specific affinities to the plurality of analytes-of-interest, each the affinity moiety having a predetermined, measurable response to light; (c) a container for combining the objects, the at least one affinity moiety and the sample under conditions for affinity binding; and (d) a determinator for simultaneously determining, for each object of the plurality of objects an imagery characteristic, and for at least a portion of the at least one affinity moiety a response to light, thereby detecting the presence, absence and/or level of the plurality of analytes-of-interest in the sample.

According to still further features in the described preferred embodiments the determinator is a spectral imaging device operable to construct a spectral image of the sample.

According to still further features in the described preferred embodiments the spectral image comprises at least two colors.

According to still further features in the described preferred embodiments the spectral image comprises at least three colors.

According to still further features in the described preferred embodiments the spectral image comprises at least four colors.

According to still further features in the described preferred embodiments the determinator is operable to determine, for each object, a wavelength value and an intensity value.

According to still further features in the described preferred embodiments the determinator is operable to determine a presence of a particular analyte of the plurality of analytes-of-interest in the sample, based on the wavelength value.

According to still further features in the described preferred embodiments the determinator is operable to determine a level of a particular analyte of the plurality of analytes-of-interest in the sample, based on the intensity value.

According to still further features in the described preferred embodiments the analytes-of-interest are dissolved, suspended or emulsed in a solution.

According to still further features in the described preferred embodiments the analytes-of-interest are selected from the group consisting of antigens, antibodies, receptors, haptens, enzymes, proteins, peptides, nucleic acids, drugs, hormones, chemicals, polymers, pathogens, toxins, and combination thereof.

According to still further features in the described preferred embodiments the analytes-of-interest are selected from the group consisting of viruses, bacteria, cells and combination thereof.

According to still further features in the described preferred embodiments the unique geometrical shape is selected from the group consisting of a spherical shape, a pyramidal shape, a flat shape and an irregular shape.

According to still further features in the described preferred embodiments a portion of the plurality of objects are beads.

According to still further features in the described preferred embodiments a portion of the plurality of objects are disks.

According to still further features in the described preferred embodiments the plurality of objects are predetermined spatial x-y locations on two-dimensional array.

According to still further features in the described preferred embodiments the two-dimensional array is a micro-array chip.

According to still further features in the described preferred embodiments the objects are of micrometer size.

According to still further features in the described preferred embodiments each of the plurality of objects comprises a predetermined combination of color-components, each color-component is selected from the group consisting of fluorochromes, chromogenes, quantum dots, nanocrystals, nanoprisms, nanobarcodes, scattering metallic objects, resonance light scattering objects and solid prisms. According to still further features in the described preferred embodiments each of the color-components is characterized by a predetermined concentration level.

According to still further features in the described preferred embodiments each of the fluorochromes is selected from the group consisting of Aqua, Texas-Red, FITC, rhodamine, rhodamine derivative, fluorescein, fluorescein derivative, cascade blue, Cyanine and Cyanine derivatives.

According to still further features in the described preferred embodiments the specific affinity of each of the plurality of objects and the specific affinity of each of the at least one affinity moiety are independently capable of binding to an analyte by means of an ionic linkage or a non-ionic linkage.

According to still further features in the described preferred embodiments the specific affinity of each of the plurality of objects and the specific affinity of each of the at least one affinity moiety are independently capable of binding to an analyte by means of covalent linkage or a non-covalent linkage.

According to still further features in the described preferred embodiments the specific affinity of each object of the plurality of objects is adsorbed onto a surface of the object.

According to still further features in the described preferred embodiments the specific affinity of each object of the plurality of objects is covalently linked to the object.

According to still further features in the described preferred embodiments the specific affinity of each of the plurality of objects and the specific affinity of each of the at least one affinity moiety are independently selected from the group consisting of a nucleic acid, an antibody, an antigen, a receptor, a ligand, an enzyme, a substrate and an inhibitor.

According to still further features in the described preferred embodiments the container comprises a plurality of x-y location on a two-dimensional platform.

According to still further features in the described preferred embodiments the two-dimensional platform is a microtiter plate.

According to still further features in the described preferred embodiments the two-dimensional platform is a microscope slide.

According to still further features in the described preferred embodiments the determinator is operable to process each x-y location separately.

According to still further features in the described preferred embodiments the determinator is operable to process all x-y locations simultaneously.

According to still further features in the described preferred embodiments the determinator is operable to simultaneously determine responses to light of the plurality of objects and responses to light of the at least one moiety.

According to still further features in the described preferred embodiments the determinator is operable to simultaneously determine responses to light of the plurality of objects and responses to light of the at least one moiety one at a time.

According to still further features in the described preferred embodiments the determinator is operable to generate a gray-level image of responses to light of the at least one moiety.

According to still further features in the described preferred embodiments the system further comprising a background subtractor for collecting and subtracting background spectra from the spectral image, the background spectra are collected from a regions of the image which are characterized by absence of objects.

According to still further features in the described preferred embodiments the system further comprising a magnifier for magnifying the spectral image by a magnification factor, the magnification factor is from 1 to 100.

According to still further features in the described preferred embodiments the system further comprising an epi-fluorescent setup which comprises at least one filter for selecting an optimal excitation and emission spectrum of each of the plurality of objects.

According to still further features in the described preferred embodiments the determinator comprises a spectral analyzer operable to perform a procedure selected from a group consisting of a principle component analysis, a principle component regression and a spectral decomposition.

According to still further features in the described preferred embodiments the determinator communicates with a library of reference spectra characterizing the plurality of objects.

According to still further features in the described preferred embodiments the interferometer is selected from the group consisting of a moving type interferometer, a Michelson type interferometer and a Sagnac type interferometer.

According to still further features in the described preferred embodiments the dispersion element is at least one filter, selected so as to collect spectral data of intensity peaks characterizing a response to light of each of the plurality of objects.

According to still further features in the described preferred embodiments each of the at least one filter is independently selected from the group consisting of an acousto-optic tunable filter and a liquid-crystal tunable filter.

According to still further features in the described preferred embodiments the dispersion element is selected from the group consisting of a grating and a prism.

According to still further features in the described preferred embodiments the detector is selected from the group consisting of a CCD detector, a C-MOS detector, a line-scan array, an array of photo diodes and a photomultiplier.

According to still further features in the described preferred embodiments the determinator comprises: (i) at least one light source for illuminating the sample with incident light; and (ii) a collector for collecting exiting light from the sample so as to acquire a spectrum of each object of the plurality of objects.

According to still further features in the described preferred embodiments the exiting light is reflected from the sample.

According to still further features in the described preferred embodiments the exiting light is transmitted through the sample.

According to still further features in the described preferred embodiments the exiting light is emitted from the sample.

According to still further features in the described preferred embodiments the system further comprising at least one filter for adjusting a spectrum of the incident light.

According to still further features in the described preferred embodiments the system further comprising an optical device for substantially filtering out an exciting wavelength of the incident light while collecting the exiting light.

According to still further features in the described preferred embodiments the optical device is selected from the group consisting of a filter, a dichroic mirror, a dark-field objective lens, a phase contrast device and a Numarski-prism.

According to still further features in the described preferred embodiments the collector is characterized by spectral resolution ranging between 1 nm and 50 nm and spatial resolution ranging between 0.1 mm and 1.0 mm.

According to still further features in the described preferred embodiments the spectral imaging device is operable to generate individual spectra-images from spectra acquired by the collector.

According to still further features in the described preferred embodiments the at least one light source is selected from the group consisting of Mercury lamp, Xenon lamp, Tungsten lamp, Halogen lamp, laser light source, Metal-Halide lamp. According to still further features in the described preferred embodiments the spectral imaging device comprises an interferometer and a detector, the interferometer comprising two mirrors and one beam-splitter, and the detector comprising a two dimensional array of detector elements.

According to still further features in the described preferred embodiments the detector is a CCD detector.

According to still further features in the described preferred embodiments the system further comprising a collimator designed and configured such that light is simultaneously collected and collimated for each of the plurality of objects.

According to still further features in the described preferred embodiments the collimator is an afocal telescope.

According to still further features in the described preferred embodiments the collimator is a microscope.

According to still further features in the described preferred embodiments the beam-splitter and the two mirrors are operable to rotate rigidly about a predetermined axis.

According to still further features in the described preferred embodiments the interferometer further comprises a first periscope mirror, a second periscope mirror and a double sided mirror having a first side and a second side, wherein the double sided mirror is operable to rotate about a predetermined axis.

According to still further features in the described preferred embodiments the interferometer further comprises a single large mirror, operable to rotate about a predetermined axis.

According to still further features in the described preferred embodiments the beam-splitter and the two mirrors are combined in a single rigid element, shaped as a prism.

According to still further features in the described preferred embodiments the beam-splitter and the two mirrors are combined in a single rigid element, shaped as a grating.

According to still further features in the described preferred embodiments the beam-splitter and the two mirrors are combined in a single rigid element, shaped as a combination of a prism and a grating.

According to still further features in the described preferred embodiments the system further comprising a transmitting unit for simultaneously transferring all data in real time from all the elements of the detector array to a computer, and displaying an image on an output device.

According to still further features in the described preferred embodiments the output device is a screen.

According to still further features in the described preferred embodiments the output device is a printed image.

The present invention successfully addresses the shortcomings of the presently known configurations by providing a method and system for the analysis of biological samples far exceeding prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows an object having a response to light and a plurality of copies of affinity moieties having a different response to light, according to the present invention;

FIG. 2 shows a possible configuration for obtaining the response to light of the object, according to the present invention;

FIG. 3a shows a first vial for storing and delivering the objects, according to the present invention;

FIG. 3b shows a second vial for storing and delivering the affinity moieties, according to the present invention;

FIG. 4 shows a measurement setup, according to the present invention;

FIG. 5 is a block diagram of the main components of an imaging spectrometer, according to prior art;

FIG. 6 shows an imaging spectrometer utilizing an interferometer having a variable optical path difference, according to prior art;

FIG. 7 shows a filters-based spectral imaging device, according to prior art;

FIG. 8 shows spectra of four different beads each having a different fluorochrome, according to the present invention;

FIGS. 9a-b show the spectral image of the four different beads, according to the present invention;

FIG. 10 shows spectra of 10 different beads labeled using combinatorial labeling, according to the present invention;

FIG. 11 shows the result of an image analysis algorithm that identifies all the beads in a spectral image, according to the present invention;

FIG. 12 shows a scatter plot of the analyzed beads spectra, according to the present invention; and

FIG. 13 is a simplified flowchart of a procedure for acquisition and data processing of a sample including a plurality of beads.

### DESCRIPTION OF THE PREFERRED EMBODIMENTS

The present invention is of a method and system for simultaneously detecting the presence, absence and/or level of a plurality of analytes-of-interest in a sample, which can be used for simultaneous biochemical studies and diagnostic tests. Specifically, the present invention can be used to simultaneously detect the presence, absence and/or level of a wide range of analytes including, but not limited to, small molecules, biopolymers, such as proteins and nucleic acids, and living organisms such as bacteria, phages, viruses, cells and the like.

The principles and operation of a method and system for simultaneously detecting the presence, absence and/or level of a plurality of analytes according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

According to one aspect of the invention there is provided a method of detecting the presence, absence and/or level (e.g., amount, concentration) of a plurality of analytes-of-interest in a sample (e.g., in an admixture of analytes). The method comprising the following method steps in which, in a first step a plurality of objects are provided, whereby each object has a predetermined, measurable and different imagery characteristic.

According to a preferred embodiment of the present invention, the different imagery characteristic may be any imagery characteristic suitable for distinguishing between two objects such as, but not limited to, a unique size, a unique geometrical shape and/or a unique response to light.

For example, an imagery characteristic which is a response to light can be uniquely quantified by a spectrum of light which may be emitted by, transmitted through or reflected from the objects.

For the purpose of simplifying the description, but without limiting the scope of the present invention, the description below first focuses on imagery characteristic which is a response to light. Other embodiments in which the imagery characteristic is, e.g., a unique size and/or a unique geometrical shape are provided hereinafter.

In addition to the different imagery characteristics, each object has a predetermined and specific affinity to one of the analytes-of-interest, so as to uniquely pair a unique imagery characteristic with a unique affinity to an analyte for each object in the population of objects. Thus, each imagery characteristic uniquely corresponds to an affinity to a specific analyte.

In a second step of the method of the present invention, at least one affinity moiety is provided, having a predetermined and specific affinity or predetermined and specific affinities to the analytes.

Each of the affinity moieties has a predetermined and measurable response to light. The affinity moieties are provided for the purpose of marking those objects that are populated by analytes and as such, both the number of different responses to light and the number of different specific affinities of the affinity moieties may vary.

Specifically, in one embodiment, all the affinity moieties are characterized by a common response to light and in another embodiment each affinity moiety is characterized by a different response to light. Additionally, one or more of the affinity moieties may have a common affinity to one or more of the analytes.

In a third step of the method, the objects, the affinity moieties and the sample are combined under conditions for affinity binding.

The affinity moieties are preferably selected so that each object which is occupied by an analyte is marked by a respective affinity moiety, which is characterized by a predetermined response to light.

Once the affinity moieties bind their analytes which are bound to the objects, resulting are structures each having a unique pairing of (i) imagery characteristic (e.g., the inherent response to light of the object) and (ii) a response to light of the affinity moiety. These structures are washed prior to the next measurement step, such that any unbound affinity moieties are removed.

In a forth step of the method of the present invention, the combination of imagery characteristic inherent to the object and the response to light inherent to the affinity moiety are detected for each object (structure), so as to determine the presence, absence and/or level of the respective analytes in the analyzed sample.

It is the combination of imagery characteristics and responses to light of both the objects and the affinity moieties bound thereto through analytes which is measured and this combination is used for determining the presence, absence (i.e., by determining the presence or absence of given combinations) and/or level (once a combination is present, level is determinable by determining the level of the response to light of the affinity moiety) of any one of the analytes-of-interest present in the analyzed sample.

According to a preferred embodiment of the present invention, the forth step may be executed by any device known in the art which is capable of measuring the responses to light of all the objects simultaneously. One known such device is a spectral imaging device which is operable to construct a spectral image for the objects. Spectral imaging methods and other methods which may be used in the detection step, according to this aspect of the present invention, are further detailed and exemplified hereinafter.

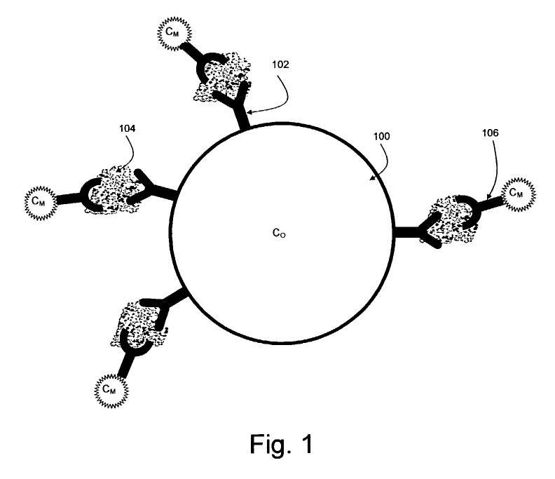

Referring now to the drawings, FIG. 1 schematically illustrates an analyte 104, an object 100 and an affinity moiety 106. Object 100 has a response to light, C_{O}, and a specific affinity 102 to analyte 104. Affinity moiety 106 may be different from specific affinity 102 provided both affinity moiety 106 and specific affinity 102 are capable of binding to the same analyte-of-interest (e.g., analyte 104). Affinity moiety 106 has a response to light, which is denoted C_{M} in FIG. 1.

Once the conditions for affinity binding have been generated, analyte 104 binds to object 100 and affinity moiety 106 binds to analyte 104 thereby marking object 100 as being occupied by analyte 104. The detection step is preferably by illuminating the sample (or a portion of the sample) by light, which is then recorded as a plurality of signals by a detecting device. A signal, coming from, e.g., object 100 and all the copies of affinity moiety 106 which are bound to object 100 through analyte 104, is a combination of C_{O} and C_{M}. Such signal preferably includes the wavelength as well as the intensity of the emerging light. The signal may also include the polarization and/or the response time of the object. The wavelengths serve as a labeling parameter, distinguishing between different combinations of C_{O} and C_{M}, thereby allowing the detection of the presence or absence of analyte 104, while the intensity serves as a level parameter, as is further explained herein.

A particular feature of a preferred embodiment of the present invention is that there is more than one location on object 100 onto which analyte 104 binds. The measured intensity is proportional to the number of copies of affinity moiety 106 which bound object 100 through analyte 104, and thereby to the concentration or amount of analyte 104 in the sample.

Thus, measuring the intensity of the light is equivalent to measuring the level of analyte 104. As there are a large number of molecules which may occupy a single object, the number of copies of affinity moiety 106 per object is also large. Thus, the physical size of object 100 is typically far larger than the physical size of affinity moiety 106. Preferably, the size difference between object 100 and affinity moiety 106 is selected so that the number of analyte molecules (and consequently affinity moiety molecules) which may occupy a single object is in the order of 10⁵-10⁷, preferably about 10⁶.

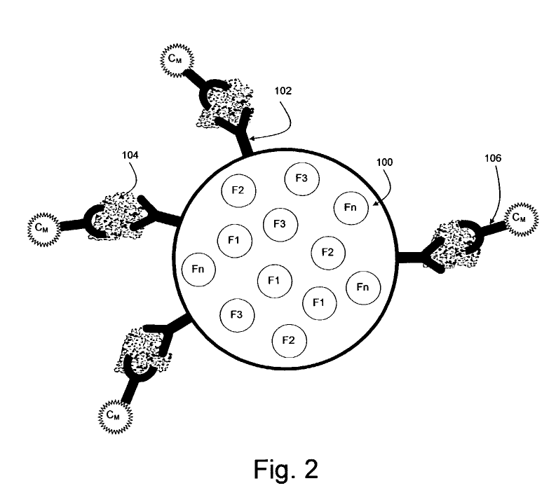

Reference is now made to FIG. 2 which illustrates a possible and non-limiting way of obtaining the response to light, C_{O}, of object 100.

Hence, according to a preferred embodiment of the present invention, the response to light C_{O} is associated with object 100 using a method known in the art as combinatorial labeling [to this end see, e.g., Ried et al., “Simultaneous Visualization of Seven Different DNA Probes by In Situ Hybridization Using Combinatorial Fluorescence and Digital Imaging Microscopy”, Proc. Natl. Acad. Sci., 1388-1392 (1992)].

Thus, object 100 preferably comprises a predetermined combination of color-components. Generally, there can be n types of color-components designated in FIG. 2 as F1, F2, . . . , Fn.

According to a preferred embodiment of the present invention, the response to light, C_{M}, can be associated to affinity moiety 106 using similar principles.

Both the number (n) and the concentration levels of the color-components are preferably selected so as to obtain the desired responses to light, C_{O}, and/or C_{M}.

A skilled artisan would appreciate that for a given n there can be many different combinations of color responses. For example, for n=3, a concentration levels ratio of F1:F2:F3=1:1:1 gives a certain response to light while a concentration levels ratio of F1:F2:F3=2:1:1 gives a different response to light. The number of different combinatorial combinations increases exponentially both with n and with the number of different concentration levels that are used.

For n types of color-components and m different levels of concentration, it is possible to achieve mⁿ−1 different responses to light. For example, with n=5 and m=5 there are 3124 different discernable spectra.

The number of different combinations of C_{O} and C_{M} is preferably larger than- or equal to the number of analytes-of-interest. As stated, there can be any number of different responses to light of affinity moiety 106. However, due to the nature of affinity moiety 106 it is preferred that there would be a larger number of different responses to light for objects 100 (many different C_{O}'s) and a smaller number of different responses to light for affinity moieties 106 (small number of C_{M}'s). For example, it may be that there is only one C_{M}, and the number of different C_{O}'s equals the number of analytes-of-interest. Thus, according to preferred embodiments of the invention, the ratio between the number of C_{O}'s and C_{M}'s is greater than 1, preferably, greater than 10.

According to a preferred embodiment of the present invention, any type of color-components can be used for providing the responses C_{M} and C_{O}. The color-components which are used according to a preferred embodiment of the present invention are not limited to any specific type. Broadly speaking, different color-components have different physical properties and different manufacturing possibilities. Many types of color-components are known in the art and are commercially available. Examples for different types of color-components are provided in the following embodiments.

Hence, in one embodiment, the color-components are fluorescent materials (fluorochromes) facilitating the fluorescence phenomenon described in the Background section above. The advantage of using fluorescent materials is that the signal is emitted only from the fluorescent materials whereas the background remains dark [to this end see, e.g., J. S. Ploem, “Introduction to Fluorescence Microscopy”, Oxford Science Publications, New York 1987; Lakowicz, “Principles Of Fluorescence Spectroscopy”, Plenum Press, New York, London, 1983]. An additional advantage of using fluorochromes is the large variety of biological structures to which specific fluorochromes can be bound [Waggoner, “Applications of Fluorescence in the Biomedical Sciences”, Eds. Taylor et al., New York; Alan R. Liss, Inc. 3-28 (1986); Mason (editor), “Fluorescent and Luminescent Probes for Biological Activity”, Biological Techniques Series, Academic Press Limited, London, (1993)].

According to a preferred embodiment of the present invention any type of fluorescent material may be used. Preferably, but not exclusively, these include fluorochromes, quantum dots or nanocrystals.

The advantages and basic characteristics of the above material are summarized herein, with references to related publications, all of which are hereby incorporated by reference.

Fluorochromes are bright, chemically stable organic materials and can be attached to different compounds and/or surfaces [Taylor et al., “The New Vision of Light Microscopy”, American Scientist 80, 322-335, (1992).

Quantum dots or nanocrystals are based on a small size semiconductor that fluoresces. Small size semiconductors are known to be much more stable than the organic-materials based fluorochromes [Bruchez et al., “Semiconductor Nanocrystals As Fluorescent Biological Labels”, Science 281:2013-2016 (1998); Chan W. C. et al., “Quantum Dot Bioconjugates For Ultra sensitive Nonisotopic Detection”, Science 281:2016-2018 (1998)]. In addition, it is possible to design and manufacture a family of narrow bandwidth nanocrystals having a common excitation wavelength, but different emitted wavelength.

According to a preferred embodiment of the present invention, other color-components which may be used include, but are not limited to, metallic bar-codes, nanoprisms, resonance light scattering particles, chromogenes, nanobarcodes, scattering metallic particles and solid prisms [to this end see, respectively, Sheila et al. “Submicrometer Metallic Barcodes”, Science 80:137-141 (2001); Rongchao J. et al., “Photoinduced Conversion of Silver Nanospheres to Nanoprisms”, Science 294:1901-1903 (2001); and Bao P. et al., “High-Sensitivity Detection of DNA Hybridization on Microarrays Using Resonance Light Scattering”, Anal Chem. 15:1792-1797 (2002)].

The various color components listed above have different physical characteristics. Depending on the type of the color-components being used, light may be transmitted through, reflected from or emitted by object 100 and affinity moiety 106.

According to a preferred embodiment of the present invention the objects (e.g., object 100) may be provided in more than one form. For example, each of the plurality of objects may be a particle of micrometric size. Unlike in prior art methods (e.g., flow cytometry), where the intensity of the received light depends on the orientation of the particle and therefore the particles have to be substantially spherical, the particles of the present invention may have any shape, such as, but not limited to, a spherical shape, a pyramidal shape, a flat shape (e.g., disks) or any irregular shape.

Similarly to the particular response to light of each object, the unique geometrical shape of the objects may also serve as a labeling parameter, distinguishing between objects having different unique geometrical shape.

Additionally, the object may be manufactured with different sizes, so that the size of the object may also be used as a discriminator between objects.

Hence, as already stated hereinabove, in addition to, or as an alternative to the response to light, the imagery characteristics of the objects may comprise the unique geometrical shapes and/or the sizes of the objects. When used in combination, these imagery characteristics, which are readily identifiable by conventional image processing algorithms increase the level of multiplexing of the system.

The use of flat objects is preferred in processes in which it is important to keep the objects in suspension at low stirring speeds. Another advantage of flat objects is their ability to provide a substantially uniform image. Flat disks are commercially available, for example from Nunc (Roskilde, Denemark) which manufactures 2D MicroHex™ nunclon™ microcarriers. It would be appreciated that flat objects are less suitable for flow based detection methods where the intensity should not depends on the orientation in space of the object.

According to a preferred embodiment of the present invention, the objects may be in the form of beads having a micrometric size, also known as microbeads. Microbeads are known in the art and are extensively used in many applications in life sciences and in medical diagnostics [see, for example, Singer, J. M., Plotz, C. M. “The Latex Fixation Test in Rheumatic Diseases: a Review” Amer J Med, 31:766-79 (1961)]. Typically, microbeads are made of polystyrene particles that are prepared by emulsion polymerization methods with a styrene monomer and potassium persulfate or benzoyl peroxide as polymerization initiator.

Small microbeads (less than 0.5 μm) are often prepared in one step followed by a cleaning step to remove detergents and inorganic salts. Larger microbeads are typically prepared in sequential steps by growing smaller microbeads with addition of styrene monomer and initiator. Following each growing step, the microbeads are washed using centrifugation.

The technology to make a series of multicolored, fluorescent microbeads with unique fluorescence characteristics is disclosed in numerous publications and patents [to this end see, e.g., U.S. Pat. No. 5,194,300 to Cheung; U.S. Pat. Nos. 4,774,189 and 5,073,498 to Schwartz; U.S. Pat. No. 4,717,655 to Fulwyler; and U.S. Pat. No. 5,723,218 to Haugland et al., all of which are hereby incorporated by reference].

According to a preferred embodiment of the present invention, the microbeads may be fluorochromed either by internal labeling or by external labeling (surface attachment). For further details regarding the fluorochromed microbeads, the reader is referred to an article by Arshady, R. entitled “Microspheres for Biomedical Applications: Preparation of Reactive and Labeled Microspheres”, published in Biomaterials, 14(1):5-15 (1993).

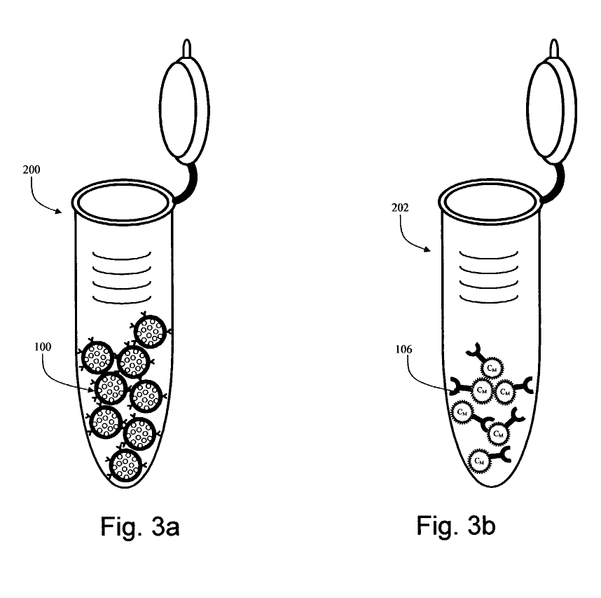

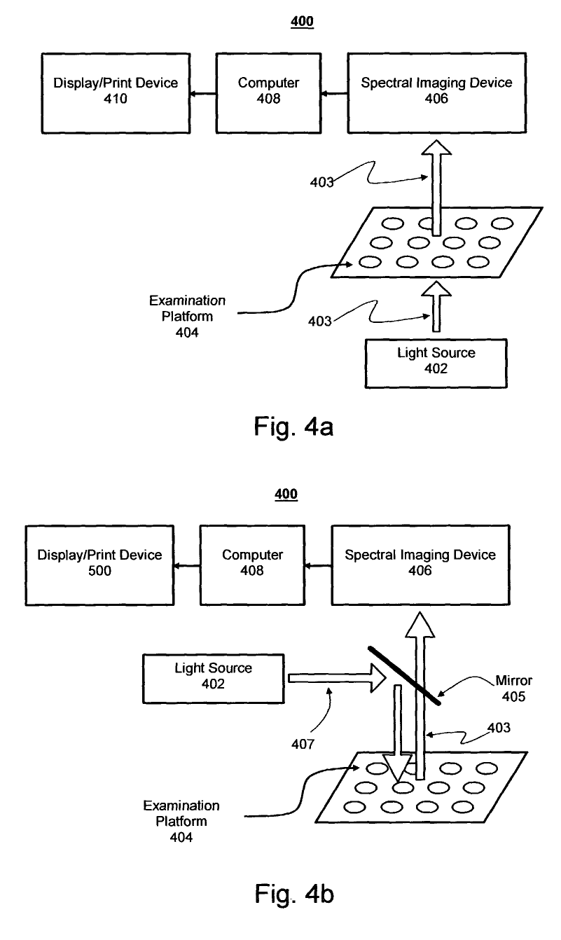

In internal labeling, a polymeric microbead is swelled in an organic fluorochrome solution. The fluorochrome diffuses into the polymer matrix, and is entrapped when the solvent is removed from the microbeads either by evaporation or by transfer to an aqueous phase. Internal labeling affords many benefits such as availability of surface groups for coupling reactions, photo-stability, protection of fluorophore from photo-bleaching, larger selection of fluorochromes and the ability to use large quantities of fluorochrome(s) per bead in order to enhance the brightness of the microbead. Reference is now made to FIGS. 3a-b, which illustrate a possible way of storing and delivering the objects and the affinity moieties, in the embodiment in which the objects are manufactured in a micro-particles (e.g., discs or beads). FIG. 3a shows a vial 200 containing only objects (such as object 100). Vial 200 may contain all the objects (i.e., many C_{O}'s in the same vial) or, alternatively, a plurality of vials, such as vial 200, can be provided whereby each vial contains a unique object. FIG. 3b shows a vial 202 containing only the affinity moieties (such as affinity moiety 106), which may have, as already explained any number of different responses to light.

According to a preferred embodiment of the present invention, the step is of combining the sample, the objects and the affinity moieties, which may be executed in any container suitable to hold the reaction mixture, is followed by positioning (e.g., by printing, or gluing) the objects on an examination platform such as, but not limited to, a microscope slide. This procedure is further exemplified in the Examples section below. A further improvement to the multiplicity of the measurement may be achieved by using a microtiter plate instead of a slide. A microtiter plate includes a plurality of wells, each of which may serve as a container for a different chemical reaction. According to a preferred embodiment of the present invention any known microtiter plate may be used, for example, a 96 wells microtiter plate, a 384 wells microtiter plate or a 3456 wells microtiter plate. It is expected, however, that during the life time of this patent other instruments will be developed and the scope of the term examination platform is intended to include all such new platforms a priori. The responses to light at each well of the plate may be redefined (i.e., a particular response to light corresponds to different specific affinities at different locations of the plate), thereby allowing more analytes to be detected at a single measurement.

In a typical process employing micro sized objects, the step of combining the objects under affinity binding condition is followed by a washing step. This may be done in more than one way. In one embodiment, the washing step is executed by evacuating the solution through a porous-type filter which keeps the objects from passing through the filter. In another embodiment, the objects are attached to the bottom of a supportive medium (e g., microtiter plates). The wash steps then executed by sucking the access material from the well while adding other solutions. As the objects are attached to the bottom, they are retained thereat through the washing procedure. Similarly, washing by immersion in a washing solution followed by centrifugation for collecting the washed objects can also be used.

According to a preferred embodiment of the present invention, the objects (such as object 100) may have forms other than micro-particles.

Hence, in another embodiment of the present invention, the objects are predetermined locations (e.g., spatial x-y locations) on a two-dimensional array, such as a micro-array chip. In this embodiment of the invention, each color-components combination, C_{O}, and each specific affinity 102 are respectively attached to a predetermined location of the two-dimensional array, and the sample and the affinity moieties (106) are added, separately, premixed or together, onto the two-dimensional array under conditions allowing affinity binding.

Irrespectively of the form in which the objects are embodied, once the sample the affinity moieties and the objects are combined, and after a sufficient number of intra-molecular interaction occurs, and following a washing step, the detection step begins.

A detailed description of the detection step according to preferred embodiments of the present invention is now provided.

Different methods are known in the art for detecting several color-components simultaneously [Garini, Y. et al., “Spectral Bio-Imaging, in Fluorescence Imaging Spectroscopy and Microscopy”, X. F. Wang and B. Herman, Editors, John Wiley and Sons, 87-124 (1996)].

When a large number of objects that are distinguishable by their response to light are used, the goal of a multi-color measurement is to unequivocally identify each one of the responses to light. As stated, in one embodiment of the invention, the responses to light of the objects are preferably effected by combinations of fluorochromes. Because the emission intensity of fluorochromes is typically a few orders of magnitude lower than the excitation intensity, it is necessary to block the excitation light from the emission path. This is done by using a set of filters in the light-path of the microscope.

In multi color measurements, several fluorochromes are used simultaneously. In order to obtain an appropriate distinction, the fluorochromes should have a spectral gap. On the other hand, the typical bandwidth of a fluorochrome spectrum (both absorption and emission) is in the range of 50-100 nm full width at half maximum and the Stokes shift is also of the same order of magnitude. In addition, the total spectral range is limited by the spectral response of the detectors and optics (typically, a spectral range of about 400-500 nm) and in order to get a sufficiently bright signal, the emission and excitation spectra of the chosen fluorochromes should fall inside these ranges. This fact results in a high degree of spectral overlap. It is this overlap that complicates the measurement of several fluorochromes simultaneously.

This major problem of spectral overlap of the fluorochromes can be overcome by performing a spectral measurement, with an appropriate selection of the fluorochromes. A fully detailed explanation of the problem that takes these aspects into account is found in a publication by Garini, Y. et al., entitled “Signal to Noise Analysis of Multiple Color Fluorescence Imaging Microscopy”, published in Cytometry, 35:214-226 (1999).

Spectral Karyotyping, which is a variant of spectral imaging, was successfully used, for example, for the detection of all the 24 different human chromosomes, each one labeled with a different combination of fluorochromes [see, Schröck, E., et al., “Multicolor Spectral Karyotyping of Human Chromosomes, Science, 273:494-7 (1996)] and led to an ever-growing usage of the method that resulted in many publication and clinical usage. A detailed review of numerous uses of Spectral Karyotyping, is found in an article by Schrock, E. et al., entitled “Spectral Karyotyping and Multicolor Fluorescence in situ Hybridization Reveal New Tumor-Specific Chromosomal Aberrations”, published in Semin. Hematol. 37:334-47 (2000).

Hence, as already mentioned hereinabove, according to a preferred embodiment of the present invention the detection step is executed by a spectral imaging device which is operable to construct a spectral image of the objects. By using a spectral imaging device in the detection step, the wavelength, the intensity of the light for each wavelength, the unique geometrical shape of the objects and/or the size of the objects can be determined simultaneously and independently. Hence, the present invention successfully provides a tool for performing multiplexed assays.

Following is a general review of spectral imaging methods and spectral images.

A spectral imaging device, also referred to herein as “imaging spectrometer”, is a spectrometer which collects incident light from a scene and measures the spectra of each picture element thereof. A spectrometer is an apparatus designed to accept light, to separate (disperse) it into its component wavelengths, and measure the lights spectrum, that is the intensity of the light as a function of its wavelength. Spectroscopy is a well known analytical tool which has been used for decades in science and industry to characterize materials and processes based on the spectral signatures of chemical constituents therein. The physical basis of spectroscopy is the interaction of light with matter. Traditionally, spectroscopy is the measurement of the light intensity emitted, scattered or reflected from or transmitted through a sample, as a function of wavelength, at high spectral resolution, but without any spatial information.

Spectral imaging, on the other hand, is a combination of high resolution spectroscopy and high resolution imaging (i.e., spatial information).

Most of the works so far described in spectral imaging concern either obtaining high spatial resolution information from a biological sample, yet providing only limited spectral information, for example, when high spatial resolution imaging is performed with one or several discrete band-pass filters [See, Andersson-Engels et al., Proceedings of SPIE—Bioimaging and Two-Dimensional Spectroscopy, 1205:179-189 (1990)], or alternatively, obtaining high spectral resolution (e.g., a full spectrum), yet limited in spatial resolution to a small number of points of the sample or averaged over the whole sample [See for example, U.S. Pat. No. 4,930,516, to Alfano et al.].

Conceptually, a spectral imaging system comprises (i) a measurement system, and (ii) an analysis software. The measurement system includes all of the optics, electronics and the manner in which the sample is illuminated (e.g., light source selection), the mode of measurement (e.g., fluorescence, transmission or reflection), as well as the calibration best suited for extracting the desired results from the measurement. The analysis software includes all of the software and mathematical algorithms necessary to analyze and display important results in a meaningful way.

Spectral imaging has been used for decades in the area of remote sensing to provide important insights in the study of Earth and other planets by identifying characteristic spectral absorption features originating therefrom. However, the high cost, size and configuration of remote sensing spectral imaging systems (e.g., Landsat, AVIRIS) has limited their use to air and satellite-born applications [See, Maymon and Neeck (1988) Proceedings of SPIE—Recent Advances in Sensors, Radiometry and Data Processing for Remote Sensing, 924:10-22; Dozier (1988) Proceedings of SPIE—Recent Advances in Sensors, Radiometry and Data Processing for Remote Sensing, 924:23-30].

There are three basic types of spectral dispersion methods that might be considered for a spectral imaging system: (i) spectral grating or prism, (ii) spectral filters and (iii) interferometric spectroscopy. As will be described below, the latter is best suited to implement the method of the present invention, yet certain filter-based configurations may also prove applicable.

In a grating or prism (i.e., monochromator) based systems, also known as slit-type imaging spectrometers, such as for example the DILOR system: [see, Valisa et al. (September 1995) presentation at the SPIE Conference European Medical Optics Week, BiOS Europe 1995, Barcelona, Spain], only one axis of a charge coupled device (CCD) array detector (the spatial axis) provides real imagery data, while a second (spectral) axis is used for sampling the intensity of the light which is dispersed by the grating or prism as function of wavelength. The system also has a slit in a first focal plane, limiting the field of view at any given time to a line of picture elements. In these systems, a full image can be obtained after scanning the grating (or prism) or the incoming beam in a direction parallel to the spectral axis of the CCD in a method known in the literature as line scanning.

Filters-based spectral dispersion methods can be further categorized into discrete filters and tunable filters. In these types of imaging spectrometers the spectral image is built by filtering the radiation for all the picture elements of the scene simultaneously at a different wavelength at a time by inserting, in succession, narrow band pass filters in the optical path, or by electronically scanning the bands using acousto-optic tunable filters (AOTF) or liquid-crystal tunable filter (LCTF), see below. Similarly to the slit type imaging spectrometers equipped with a grating or prism as described above, while using filters-based spectral dispersion methods, most of the radiation is rejected at any given time. In fact, the measurement-of the whole image at a specific wavelength is possible because all the photons outside the instantaneous wavelength being measured are rejected and do not reach the CCD.

Tunable filters, such as AOTFs and LCTFs have no moving parts and can be tuned to any particular wavelength in the spectral range of the device in which they are implemented. One advantage of using tunable filters as a dispersion method for spectral imaging is their random wavelength access; i.e., the ability to measure the intensity of an image at a number of wavelengths, in any desired sequence without the use of filter wheels.

A method and apparatus for spectral analysis of images which have advantages in the above respects is disclosed in U.S. Pat. No. 5,539,517, the contents of which are hereby incorporated by reference, with the objective to provide a method and apparatus for spectral analysis of images which better utilizes all the information available from the collected incident light of the image to substantially decrease the required frame time and/or to substantially increase the signal-to-noise ratio, as compared to the conventional slit- or filter type imaging spectrometer, and does not involve line scanning. According to this invention, there is provided a method of analyzing an optical image of a scene to determine the spectral intensity of each picture element (i.e., region in the field of view which corresponds to a pixel in an image presenting same) thereof by collecting incident light from the scene; passing the light through an interferometer which outputs modulated light corresponding to a predetermined set of linear combinations of the spectral intensity of the light emitted from each picture element; focusing the light outputted from the interferometer on a detector array, scanning the optical path difference (OPD) generated in the interferometer for all picture elements independently and simultaneously and processing the outputs of the detector array (the interferograms of all picture elements separately) to determine the spectral intensity of each picture element thereof.

This method may be practiced by utilizing various types of interferometers wherein the optical path difference (OPD) is varied to build the interferograms by moving the entire interferometer, an element within the interferometer, or the angle of incidence of the incoming radiation. In all of these cases, when the scanner completes one scan of the interferometer, the interferograms for all picture elements of the scene are completed.

Apparatuses in accordance with the above features differ from the conventional slit- and filter type imaging spectrometers by utilizing an interferometer as described above, therefore not limiting the collected energy with an aperture or slit or limiting the incoming wavelength with narrow band interference or tunable filters, thereby substantially increasing the total throughput of the system. Thus, interferometer-based apparatuses better utilize all the information available from the incident light of the scene to be analyzed, thereby substantially decreasing the measurement time and/or substantially increasing the signal-to-noise ratio (i.e., sensitivity). The sensitivity advantage that interferometric spectroscopy has over the filter and grating or prism methods is known in the art as the multiplex or Fellgett advantage [see, Chamberlain “The principles of interferometric spectroscopy”, John Wiley and Sons, pp. 16-18 and p. 263 (1979)].

In U.S. Pat. No. 5,748,162, which is incorporated by reference as if fully set forth herein, the objective was to provide spectral imaging methods for biological research, medical diagnostics and therapy, which methods can be used to detect spatial organization (ie., distribution) and to quantify cellular and tissue natural constituents, structures, organelles and administered components such as tagging probes (e.g., fluorescent probes) and drugs using light transmission, reflection, scattering and fluorescence emission strategies, with high spatial and spectral resolutions.

Other uses of the spectral imaging device described in U.S. Pat. No. 5,539,517 are described in the U.S. Patent Nos. 6,088,099 “Method for interferometer based spectral imaging of moving objects”, 6,075,599 “Optical device with entrance and exit paths that are stationary under device rotation”, 6,066,459 “Method for simultaneous detection of multiple fluorophores for in situ hybridization and multicolor chromosome painting and banding”; U.S. Pat. No. 6,055,325 “Color display of chromosomes or portions of chromosomes” U.S. Pat. No. 5,043,039 “Method of and composite for in situ fluorescent hybridization” U.S. Pat. No. 6,018,587 “Method for remote sensing analysis be decorrelation statistical analysis and hardware therefore”; U.S. Pat. No. 6,007,996 “In situ method of analyzing cells”; U.S. Pat. No. 5,995,645 “Method of cancer cell detection”; U.S. Pat. No. 5,991,028 Spectral bio-imaging methods for cell classification”; U.S. Pat. No. 5,936,731 “Method for simultaneous detection of multiple fluorophores for in situ hybridization and chromosome painting”; U.S. Pat. No. 5,912,165 “Method for chromosome classification by decorrelation statistical analysis and hardware therefore”; U.S. Pat. No. 5,906,919 “Method for chromosomes classification”; U.S. Pat. No. 5,871,932 “Method of and composite for fluorescent in situ hybridization”; U.S. Pat. No. 5,856,871 “Film thickness mapping using interferometric spectral imaging”; U.S. Pat. No. 5,835,214 “Method and apparatus for spectral analysis of images”; U.S. Pat. No. 5,834,203 “Method for classification of pixels into groups according to their spectra using a plurality of wide band filters and hardware therefore”; U.S. Pat. No. 5,817,462 “Method for simultaneous detection of multiple fluorophores for in situ hybridization and multicolor chromosome painting and banding”; U.S. Pat. No. 5,798,262 “Method for chromosomes classification”; U.S. Pat. No. 5,784,162 “Spectral bio-imaging methods for biological research, medical diagnostics and therapy”; U.S. Pat. No. 5,719,024 “Method for chromosome classification by decorrelation statistical analysis and hardware therefore, all of which are incorporated herein by reference.

In sharp contrast to the flow cytometry method, in spectral imaging the objects are static in the image for as much as needed. Therefore, it is possible to measure smaller signals by exposing the detectors for periods of time that are as long as needed. Available CCD's allow integrating signal on the chip for periods that are in the range of milliseconds to hundreds of seconds. For long exposure times (typically longer than 2-5 seconds) the CCD is preferably cooled so as to reduce the dark noise. Many commercially available cooled CCD's provide cooling of the CCD chip either by Pletier cooling or even liquid nitrogen (see for example Roper Scientific, Tucson, Ariz. USA and Hamamatsu, Hamamatsu Japan).

Another advantage of spectral imaging is the ability to obtain more than one measurement for a given sample. This allows to first have a first image in order to determine an optimal exposure time, and then to make the actual measurement. As a skilled artisan would appreciate, in flow-based methods (e.g., flow cytometry), the only flexibility that exist is in the gain factor of the detector, and it must be determined prior to the measurement. Moreover, the gain factor is not always a linear parameter unlike the exposure time which is a natural time linear parameter.

The ability to obtain more than one measurement for a given sample may also be exploited to improve the dynamic range of the measurement. This can be done, for example, by using a different exposure time for each image. Since high signals are efficiently measured with the short exposure times while the low signals are efficiently measured through long exposure times, a plurality of measurements, each with a different exposure time, allows for detecting both high and low signals.

Hence, according to a preferred embodiment of the present invention, the responses to light of the objects can be measured in one image and the responses to light of the affinity moieties can be measured in a different image. This allows a better detection of all responses to light since the signals from the objects are typically higher than the signals from the affinity moieties. Thus, the high signals are measured using a short exposure time and the signals from the affinity moieties are measured using a longer exposure time.

According to a preferred embodiment of the present invention some of the optical elements that are used in between the two measurements, may change to increase efficiency.

It should be understood that flow-based methods lack the ability to perform subsequent measurements because of the limited time that the system has to detect the signal while the sample passes through the examination zone.

According to a preferred embodiment of the present invention few measurement results of the same image may be averaged so as to improve the signal to noise ratio.

It is therefore appreciated that spectral imaging systems are useful in providing a large amount of details where subtle spectral differences exist between spatially distributed chemical constituents.

It should be understood that the present invention is not limited to use any specific spectral imaging device, and the detection step of the present invention can be carried out using any spectral imaging device, inter alia the spectral imaging device disclosed in U.S. Pat. No. 5,539,517.

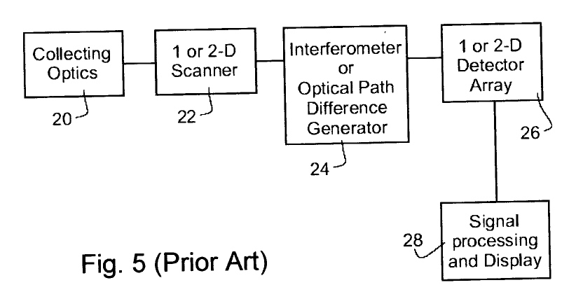

Reference is now made to FIGS. 4a-b, which illustrates a measurement setup 400, which can be used in the detection step, according to a preferred embodiment of the present invention.

An examination platform 404 that carries the objects (either in the embodiment in which the objects are micro sized objects or in the embodiment in which the objects are x-y locations on a two-dimensional array) placed in the optical path 403 of the setup.

FIG. 4a illustrates a setup which can be used in the embodiments in which the light passes through the sample. Such a setup can be adequate for color bodies such as chromogenes, each one of which absorbs a different spectrum and therefore the transmitted spectrum for each one is unique. In this embodiment, measurement setup 400 further includes a light source 402 and a spectral imaging device 406, which is communicating with a computer 408 and a display and/or printing device 410.

FIG. 4b illustrates a setup which can be used in the embodiments in which the light is emitted by or reflected from the objects, for example, in the case where the color-components are fluorochromes or in the case where the color-components are reflective (e.g., metallic disks). In both cases, this method is similar to an epi-fluorescence method where the excitation light and detection are performed from the same side of the sample (top side in FIG. 4b). In this embodiment measurement setup 400 further includes a mirror 405, positioned in optical path 403.

If the color-components are fluorochromes, then mirror 405 is preferably a dichroic mirror and other filters may be added on excitation path 407 and emission path 403 in order to ensure the elimination of the exciting light from emission path 403, while selecting the preferred spectral range for the excitation.

If the color-components are reflective, then the mirror may be part of a more complex optical setup that may include, for example, a dark-field objective lens that transmits only the light that is reflected from the color-components and absorbs the scattered light.

Irrespective of the type of objects and/or color-components which are used, spectral imaging device 406 measures the intensity levels at a certain number of spectral bands that are selected to provide the optimal ability to distinguish between objects. Spectral imaging device 406 is controlled by computer 408 which also performs an analysis of the signals as collected by spectral imaging device 406. The analyzed data are then outputted to display and/or printing device 410 which may be any known device that allows the user to make use of the data such as, but not limited to, a monitor, a printer or the like.

The following provides several alternative configurations for spectral imaging device 406. One alternative relates to interferometer-based spectral imaging devices, whereas the other relates to filters-based spectral imaging 25 devices.

Interferometer-Based Spectral Imaging Devices

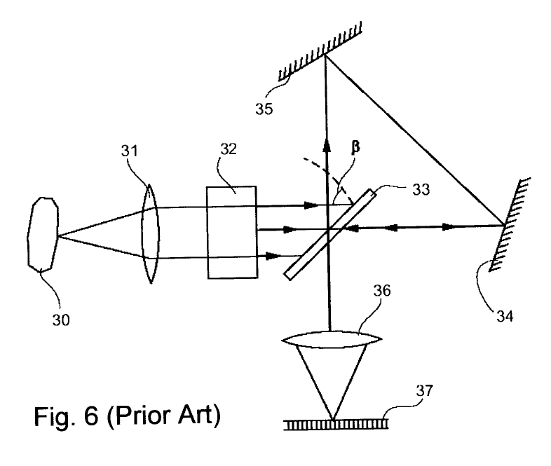

FIG. 5 is a block diagram illustrating the main components of a prior art imaging spectrometer disclosed in U.S. Pat. No. 5,539,517, which is incorporated by reference as if fully set forth herein.

This imaging spectrometer is constructed highly suitable to implement the method of the present invention as it has high spectral (Ca. 4-14 nm depending on wavelength) and spatial (Ca. system MTF (modulation transfer function, e.g., 30)/M μm, where M is the effective fore optics magnification) resolutions.

Thus, the prior art imaging spectrometer of FIG. 5 includes: a collection optical system, generally designated 20; a one-dimensional scanner, as indicated by block 22; an optical path difference (OPD) generator or interferometer, as indicated by block 24; a one-dimensional or two-dimensional detector array, as indicated by block 26; and a signal processor and display, as indicated by block 28.

A critical element is the OPD generator or interferometer 24, which outputs modulated light corresponding to a predetermined set of linear combinations of the spectral intensity of the light emitted from each picture element of the scene to be analyzed. The output of the interferometer is focused onto the detector array 26. Thus, all the required optical phase differences are scanned simultaneously for all the picture elements of the field of view, in order to obtain all the information required to reconstruct the spectrum. The spectra of all the picture elements in the scene are thus collected simultaneously with the imaging information, thereby permitting analysis of the image in a real-time manner.

The apparatus according to U.S. Pat. No. 5,539,517 may be practiced in a large variety of configurations. Specifically, the interferometer used may be combined with other mirrors as described in the relevant Figures of U.S. Pat. No. 5,539,517.

Thus, alternative types of interferometers may be employed. These include (i) a moving type interferometer in which the OPD is varied to modulate the light, namely, a Fabry-Perot interferometer with scanned thickness; (ii) a Michelson type interferometer which includes a beamsplitter receiving the beam from an optical collection system and a scanner, and splitting the beam into two paths; (iii) a Sagnac interferometer optionally combined with other optical means in which interferometer the OPD varies with the angle of incidence of the incoming radiation, such as the four-mirror plus beamsplitter interferometer as further described in the cited U.S. Pat. No. (see FIG. 14 there).

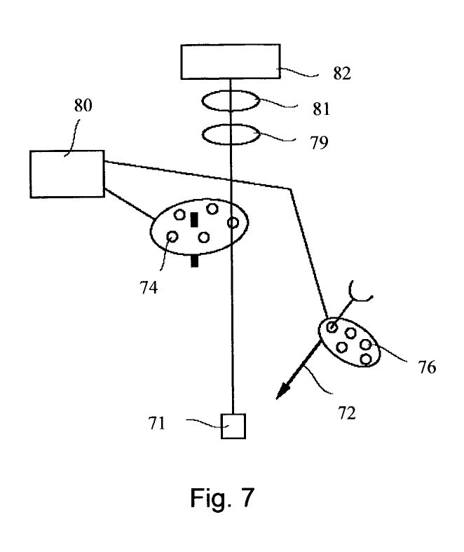

FIG. 6 illustrates an imaging spectrometer constructed in accordance with U.S. Pat. No. 5,539,517, utilizing an interferometer in which the OPD varies with the angle of incidence of the incoming radiation. A beam entering the interferometer at a small angle to the optical axis undergoes an OPD which varies substantially linearly with this angle.

In the interferometer of FIG. 6, all the radiation from source 30 in all the picture elements, after being collimated by an optical collection system 31, is scanned by a mechanical scanner 32. The light is then passed through a beamsplitter 33 to a first reflector 34 and then to a second reflector 35, which reflects the light back through the beamsplitter 33 and then through a focusing lens 36 to an array of detectors 37 (e.g., a CCD). This beam interferes with the beam which is reflected by 33, then by second reflector 35, and finally by first reflector 34.

At the end of one scan, every picture element has been measured through all the OPD's, and therefore the spectrum of each picture element of the scene can be reconstructed by Fourier transformation. A beam parallel to the optical axis is compensated, and a beam at an angle, θ, to the optical axis undergoes an OPD correction, which is a function of the thickness of the beamsplitter 33, its index of refraction, and the angle θ. The OPD is proportional to sinθ, hence to θ for small angles. By applying the appropriate inversion, and by careful bookkeeping, the spectrum of every picture element is calculated.

In the configuration of FIG. 6 the ray which is incident on the beamsplitter at an angle β(β=45° in FIG. 6) goes through the interferometer with an OPD=0, whereas a ray which is incident at a general angle β−θ undergoes an OPD given by Equation (1):

OPD(β,θ,t,n)=t[(n²−sin²(β+θ))^{0.5}−(n²−sin²(β−θ))^{0.5}+2 sinβsin θ] (1)

where θ is the angular distance of a ray from the optical axis or interferometer rotation angle with respect to the central position; t is the thickness of the beamsplitter; and n is the index of refraction of the beamsplitter.

It follows from the above equation that by scanning both positive and negative angles with respect to the central position, one gets a double-sided interferogram for every picture element, which helps eliminate phase errors giving more accurate results in the Fourier transform calculation. The scanning amplitude determines the maximum OPD reached, which is related to the spectral resolution of the measurement. The size of the angular steps determines the OPD step which is, in turn, dictated by the shortest wavelength to which the system is sensitive. In fact, according to the sampling theorem [see, Chamberlain (1979) “The principles of Interferometric Spectroscopy”, John Wiley and Sons, pp. 53-55], this OPD step must be smaller than half the shortest wavelength to which the system is sensitive.

Another parameter which should be taken into account is the finite size of a detector element in the matrix. Through the focusing optics, the element subtends a finite OPD in the interferometer which has the effect of convolving the interferogram with a rectangular function. This brings about, as a consequence, a reduction of system sensitivity at short wavelengths, which drops to zero for wavelengths equal to or below the OPD subtended by the element. For this reason, one must ensure that the modulation transfer function (MTF) condition is satisfied, i.e., that the OPD subtended by a detector element in the interferometer must be smaller than the shortest wavelength at which the instrument is sensitive.

Thus, imaging spectrometers constructed in accordance with the invention disclosed in U.S. Pat. No. 5,539,517 do not merely measure the intensity of light coming from every picture element in the field of view, but also measure the spectrum of each picture element in a predefined wavelength range. They also better utilize all the radiation emitted by each picture element in the field of view at any given time, and therefore permit, as explained above, a significant decrease in the frame time and/or a significant increase in the sensitivity of the spectrometer. Such imaging spectrometers may include various types of interferometers and optical collection and focusing systems, and may therefore be used in a wide variety of applications.

An imaging spectrometer in accordance with the invention disclosed in U.S. Pat. No. 5,539,517 was developed by Applied Spectral Imaging Ltd., Industrial Park, Migdal Haemek, Israel and is referred to herein as SPECTRACUBE. This spectral imaging device was used to reduce the present invention to practice, yielding unexpected results as is further demonstrated in the Examples section that follows.

The SPECTRACUBE system has the following or better characteristics, listed hereinbelow in Table 1:

TABLE 1

| Parameter | Performance |
|---|---|
| Spatial resolution | MTF/M μm |
| | (M = effective fore opticsmagnification) |
| Field of View | 8.5/M millimeters |
| Sensitivity | 20 milliLux |
| | (for 100 msec integration time, |
| | increases for longer integration times linearly |
| | with \sqrt{T}) |
| Spectral range | 400-1000 nm |
| Spectral | 4 nm at 400 nm (16 nm at 800 nm) |
| resolution | |
| Acquisition time | 5-50 sec, typical 20 seconds |
| FFT processing | 5-60 sec, typical 20 seconds |
| time | |

Other Spectral Imaging Devices

The SPECTRACUBE system optically connected to a suitable fore optics is preferably used to analyze the objects and the affinity moieties (such as object 100 and affinity moiety 116). It would be appreciated, however, that any spectral imaging device, i.e., an instrument that measures and stores in memory for later retrieval and analysis the spectrum of light emitted by every point of an object which is placed in its field of view, including filter (e.g., conventional interference filters, acousto-optic tunable filters (AOTF) or liquid-crystal tunable filter (LCTF)) and dispersive (monochromator) element (e.g., grating or prism) based spectral imaging devices, or other spectral data or multi-band light collection devices (e.g., a device in accordance with the disclosure in an article by Speicher R. M., Ballard S. G. and Ward C. D. entitled “Karyotyping human chromosomes by combinatorial multi-flour FISH”, published in 1996 in Nature Genetics, 12:368-375) can potentially be used to acquire the required spectral data. Also a device including a plurality of wide-band of (fixed or tunable) filters, as described in U.S. Pat. No. 5,834,203, and is incorporated by reference as if fully set forth herein, can be used as the spectral data collection device according to the present invention. Therefore, it is intended not to limit the scope of the present invention for use of any specific type of spectral imaging device.

Interference Filters-Based Spectral Imaging Devices

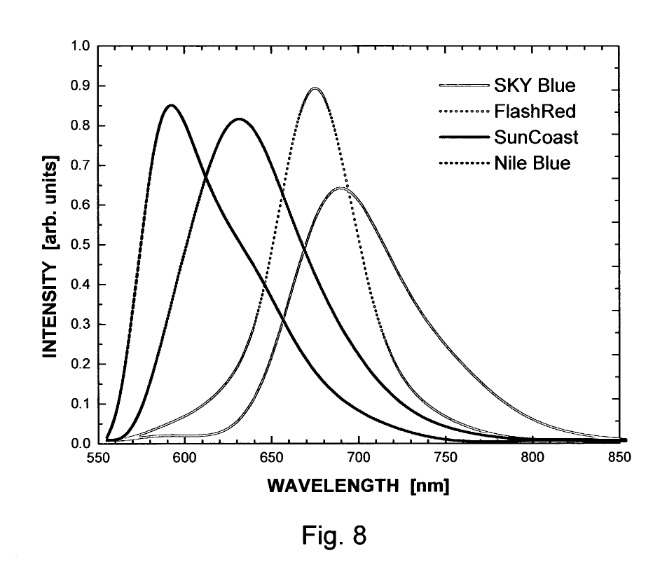

With reference now to FIG. 7. A filters-based spectral imaging device is referred to herein as apparatus 70 and includes an objective or fore optics 71. Apparatus 70 further includes a plurality of interference filters 74, five are shown. The filters are selected according to the features described hereinunder. Illumination filters 76 may also be employed, so as to restrict the illumination provided by a light beam 72 to specific wavelengths.

Apparatus 70 further includes an automatic, manual or semi-manual control device 80. Device 80 serves for selecting among filters 74 and/or 76.

Apparatus 70 further includes a light intensity recording device 82 (e.g., a CCD) which serves for recording reflected light intensity as retrieved after passing through any one of filter 74.

As a result, each of the picture elements in the analyzed sample is representable by a vector of a plurality of dimensions, the number of dimensions being equal to the number of filters 74.

In a preferred embodiment apparatus 70 further includes a collimating lens 79 to ensure fill collimation of the light before reaching recording device 82.

In a preferred embodiment apparatus 70 further includes a focusing lens 81 for focusing light reaching recording device 82.

The following provides considerations relating to filters 74 employed with apparatus 70.

Thus, according to a preferred embodiment of the present invention the filters are selected so as to collect spectral data of intensity peaks and/or steeps characterizing one or more combinations of C_{O} and C_{M}. Alternatively, filters may be selected so as to collect spectral data of intensity peaks and/or steeps characterizing a single or an averaged picture element of the sample analyzed. In any case, the normalized intensities measured using each of the discrete filters can be used as input for the algorithm of the present invention which is further described hereinunder. Thus, choice of filters is dictated by the spectral qualities one wishes to capture. The exact wavelength in which these phenomena will be detected will differ from system to system as a function of the system response. The response is composed of the CCD quantum efficiency curve, the illumination curve and the transmittance curve of the system optics.

According to preferred embodiments of the invention, each of the filters individually has a bandwidth of about 5 to about 100 nm, preferably about 10 nm, full-width-at-half-maximum filter. It will be appreciated that multiple chroic filter, such as dichroic filter or trichroic filter can replace a pair or triad of monochroic filters.

It will further be appreciated that different choices of filters are reasonable as well.

Analyzing and Displaying Spectral Imaging Data:

General Considerations and Approaches

General: A spectral image is a three dimensional array of data, I(x, y ,λ), that combines spectral information with spatial organization of the image. As such, a spectral image is a set of data called a spectral cube, due to its dimensionality, which enables the extraction of features and the evaluation of quantities that are difficult, and in some cases even impossible, to obtain otherwise. Since both spectroscopy and digital image analysis are well known fields that are covered by an enormous amount of literature [see, for example, Jain (1989) “Fundamentals of Digital Image Processing”, Prentice-Hall International], the following discussion will focus primarily on the benefit of combining spectroscopic and imaging information in a single data set, i.e., a spectral cube. Such a spectral cube of data can be collected by any spectral imaging device as is further delineated hereinabove.

One possible type of analysis of a spectral cube is to use spectral and spatial data separately, i.e. to apply spectral algorithms to the spectral data and two-dimensional image processing algorithms to the spatial data.

As an example of a spectral algorithm, consider an algorithm computing the similarity between a reference spectrum and the spectra of all pixels (i.e., similarity mapping) resulting in a gray (or other color) scale image (i.e., a similarity map) in which the intensity at each pixel is proportional to the degree of “similarity”. This gray scale image can then be further analyzed using image processing and computer vision techniques (e.g., image enhancement, pattern recognition, etc.) to extract the desired features and parameters. In other words, similarity mapping involves computing the integral of the absolute value of the difference between the spectrum of each pixel of the spectral image with respect to a reference spectrum (either previously memorized in a library, or belonging to a pixel of the same or other spectral image), and displaying a gray level or pseudocolor (black and white or color) image, in which the bright pixels correspond to a small spectral difference, and dark pixels correspond to a large spectral difference, or vice versa.

Similarly, classification mapping perform the same calculation as described for similarity mapping, yet takes several spectra as reference spectra, and paints each pixel of the displayed image with a different predetermined pseudocolor, according to its classification as being most similar to one of the several reference spectra.

It is also possible to apply spectral image algorithms based on non-separable operations; i.e., algorithms that include both local spectral information and spatial correlation between adjacent pixels (one of these algorithms is, as will be seen below, a principal component analysis).

One of the basic needs that arise naturally when dealing with any three-dimensional (3D) data structure such as a spectral cube (i.e., I(x,y,λ)), is visualizing that data structure in a meaningful way. Unlike other types of 3D data such as topographic data, D(x,y,z), obtained for example by a confocal microscope, where each point represents, in general, the intensity at a different location (x,y,z) in a tree-dimensional space, a spectral image is a sequence of images representing the intensity of the same two-dimensional plane (i.e., the sample) at different wavelengths. For this reason, the two most intuitive ways to view a spectral cube of data is to either view the image plane (spatial data) or the intensity of one pixel or a set of pixels as function of wavelength in a three-dimensional mountain-valley display. In general, the image plane can be used for displaying either the intensity measured at any single wavelength or the gray scale image that results after applying a spectral analysis algorithm, over a desired spectral region, at every image pixel. The spectral axis can, in general, be used to present the resultant spectrum of some spatial operation performed in the vicinity of any desired pixel (e.g., averaging the spectrum).

It is possible, for example, to display the spectral image as a gray scale image, similar to the image that might be obtained from a simple monochrome camera, or as a multicolor image utilizing one or several artificial colors to highlight and map important features. Since such a camera simply integrates the optical signal over the spectral range (e.g., 400 nm to 760 nm) of the CCD array, the ‘equivalent’ monochrome CCD camera image can be computed from the 3D spectral image data base by integrating along the spectral axis, as follows:

$$gray\_scale ( x , y ) = {\int }_{λ1}^{λ2} w ( λ ) \cdot I ( x , y , λ ) ⅆ λ \qquad ( 2 )$$

In Equation 2, w(λ) is a general weighting response function that provides maximum flexibility in computing a variety of gray scale images, all based on the integration of an appropriately weighted spectral image over some spectral range. For example, by evaluating Equation 2 with three different weighting functions, {wᵣ(λ), w_{g}(λ), w_{b}(λ)}, corresponding to the tristimulus response functions for red (R), green (G) and blue (B), respectively, it is possible to display a conventional RGB color image. It is also possible to display meaningful non-conventional (pseudo) color images. Consider choosing {wᵣ, w_{g}, w_{b}} to be Gaussian functions distributed “inside” a spectrum of interest, the resulting pseudo-color image that is displayed in this case emphasizes only data in the spectral regions corresponding to the weighting functions, enabling spectral differences in these three regions to be detected more clearly.

Point operations: Point operations are defined as those that are performed on single pixels, (ie., do not involve more than one pixel at a time). For example, in a gray scale image, a point operation can be one that maps the intensity of each pixel (intensity function) into another intensity according to a predetermined transformation function. A particular case of this type of transformation is the multiplication of the intensity of each pixel by a constant. Additional examples include similarity and classification mapping as described hereinabove.

The concept of point operations can also be extended to spectral images: here each pixel has its own intensity function (spectrum), i.e., an n-dimensional vector V₁(λ); λε[λ₁, λₙ]. A point operation applied to a spectral image can be defined as one that maps the spectrum of each pixel into a scalar (i.e., an intensity value) according to a transformation function:

ν₂=g(V₁(λ)); λε[λ₁, λₙ] (3)

Building a gray scale image according to Equation 3 is an example of this type of point operation. In the more general case, a point operation maps the spectrum (vector) of each pixel into another vector according to a transformation function:

V₂(l)=g(V₁(λ)); lε[1, N], λε[λ₁, λₙ] (4),

where N≦n.

In this case a spectral image is transformed into another spectral image.

One can now extend the definition of point operations to include operations between corresponding pixels of different spectral images. An important example of this type of algorithm is optical density analysis. Optical density is employed to highlight and graphically represent regions of an object being studied spectroscopically with higher dynamic range than the transmission spectrum. The optical density is related to transmission by a logarithmic operation and is therefore always a positive function. The relation between the optical density and the measured spectra is given by Lambert Beer law:

$$OD ( λ ) = - {log}_{10} \frac{I ( λ )}{{I}_{0} ( λ )} = - {log}_{10} τ ( λ ) \qquad ( 5 )$$

where OD(λ) is the optical density as a function of wavelength, I(λ) is the measured spectrum, I_{O}(λ) is a measured reference spectrum, and τ(λ) is the spectral transmittance of the sample. Equation 5 is calculated for every pixel for every wavelength where I_{O}(λ) is selected from (1) a pixel in the same spectral cube for which OD is calculated; (2) a corresponding pixel in a second cube; and (3) a spectrum from a library.

Note that the optical density does not depend on either the spectral response of the measuring system or the non-uniformity of the CCD detector. This algorithm is useful to map the relative concentration, and in some cases the absolute concentration of absorbers in a sample, when their absorption coefficients and the sample thickness are known.

Additional examples include various linear combination analysis, such as, but not limited to, (i) applying a given spectrum to the spectrum of each of the pixels in a spectral image by an arithmetical function such as addition, subtraction, multiplication division and combinations thereof to yield a new spectral cube, in which the resulting spectrum of each pixel is the sum, difference, product ratio or combination between each spectrum of the first cube and the selected spectrum; and (ii) applying a given scalar to the spectra of each of the pixels of the spectral image by an arithmetical function as described above.

Such linear combinations may be used, for example, for background subtraction in which a spectrum of a pixel located in the background region is subtracted from the spectrum of each of the pixels; and for a calibration procedure in which a spectrum measured prior to sample analysis is used to divide the spectrum of each of the pixels in the spectral image.

Another example includes a ratio image computation and display as a gray level image. This algorithm computes the ratio between the intensities at two different wavelengths for every pixel of the spectral image and paints each of the pixels in a lighter or darker artificial color accordingly. For example, it paints the pixel bright for high ratio, and dark for low ratio (or the opposite), to display distributions of spectrally sensitive materials.

Spatial-spectral combined operations: In all of the spectral image analysis methods mentioned above, algorithms are applied to the spectral data. The importance of displaying the spectrally processed data as an image is mostly qualitative, providing the user with a useful image. It is also possible, however, depending on the application, to use the available imaging data in even more meaningful ways by applying algorithms that utilize the spatial-spectral correlation that is inherent in a spectral image. Spatial-spectral operations represent the most powerful types of spectral image analysis algorithms. As an example, consider the following situation:

A sample contains k cell types stained with k different stains (the term “cell” here is used both for a biological cell, and also as “a region in the field of view of the instrument”). Each stain has a distinct spectrum and binds to only one of the k cell types. It is important to find the average intensity per cell for each one of the k cell types. To achieve this task the following procedure can be used: (i) classify each pixel in the image as belonging to one of k+1 classes (k cell types plus a background) according to its spectrum; (ii) segment the image into the various cell types and count the number of cells from each type; and (iii) sum the spectral energy contributed by each class, and divide it by the total number of cells from the corresponding class.

This procedure makes use of both spectral and spatial data. The relevant spectral data takes the form of characteristic cell spectra (i.e., spectral “signatures”), while the spatial data consists of data about various types of cells (i.e., cell blobs) many of which appear similar to the eye. In the above situation, cells can be differentiated by their characteristic spectral signature. Hence, a suitable point operation will be performed to generate a synthetic image in which each pixel is assigned one of k+1 values. Assuming that the spectra of the different cell types are known to be sᵢ(λ); i=1, 2, . . . , k, λε[λ₁, λₙ], and the measured spectrum at each pixel (x, y) is s_{x,y}(λ), λε[λ₁, λₙ], then the following algorithm is a possible method of classification:

Let e²ᵢ be the deviation of the measured spectrum from the known spectrum of the stain attached to cell type i. Then, adopting a least-squares “distance” definition, one can write:

$${e}_{i}^{2} = {\sum }_{λ ∈ {R}_{λ}}^{} {( s ( λ ) - {s}_{i} ( λ ) )}^{2} \qquad ( 6 )$$

where R_{λ} is the spectral region of interest. Each point [pixel (x, y)] in the image can then be classified into one of the k+1 classes using the following criterion:

point(x,y)εclass k+1 if e²ᵢ>threshold for all i ε[1,k]

whereas

$$\begin{matrix}\begin{matrix}point ( x , y ) ∈ class ρ if : \\ {e}^{2} i < threshold , and \end{matrix} \\ ρ is such that min [ {e}^{2} i ] = {e}^{2} ρ\end{matrix} \qquad ( 7 )$$

Steps ii and iii above (image segmentation and calculation of average intensity) are now straight-forward using standard computer vision operations on the synthetic image created in accordance with the algorithm described in Equations 6 and 7.

Another approach is to express the measured spectrum s_{x,y}(λ) at each pixel as a linear combination of the k known fluorescence spectra sᵢ(λ); i=1, 2, . . . , k. In this case one would find the coefficient vector C=[c₁, c₂, . . . , cₖ] that solves:

$$F = min {\sum }_{λ ∈ {R}_{λ}}^{} {( s ( λ ) - \hat{s} ( λ ) )}^{2} \\ where \hat{s} ( λ ) = {\sum }_{i = 1}^{k} {c}_{i} \cdot {s}_{i} ( λ ) , \qquad ( 8 )$$

where

Solving for

$$\frac{ⅆ F}{ⅆ {c}_{i}} = 0 ;$$

for i=1,2, . . . ,k (i.e., find values of cᵢ which minimize F) yields the matrix Equation:

C=A⁻¹B, (9)

where A is a square matrix of dimension k with elements:

$${a}_{m , n} = [ {\sum }_{λ ∈ {R}_{λ}}^{} {s}_{m} ( λ ) \cdot {s}_{n} ( λ ) ] , \qquad ( 10 )$$

and B is a vector defined as:

$${b}_{m} = [ {\sum }_{λ ∈ {R}_{λ}}^{} {s}_{m} ( λ ) \cdot s ( λ ) ] , m , n = 1 , 2 , , k . \qquad ( 11 )$$

Arithmetic operations may similarly be applied to two or more spectral cubes and/or spectra of given pixels or from a library. For example consider applying an arithmetic operations between corresponding wavelengths of corresponding pairs of pixels belonging to a first spectral cube of data and a second spectral cube of data to obtain a resulting third spectral cube of data for the purpose of, for example, averaging two spectral cubes of data, time changes follow-up, spectral normalization, etc.

In many cases objects present in a spectral image differ from one another in chemical constituents and/or structure to some degree, especially when stained. Using a decorrelation analysis, such as a principal component analysis, by producing covariance or a correlation matrix, enhances these differences. Decorrelation statistical analysis is directed at extracting decorrelated data out of a greater amount of data, and average over the correlated portions thereof. There are a number of related statistical decorrelation methods. Examples include but not limited to principal component analysis (PCA), canonical variable analysis and singular value decomposition, etc., of these methods PCA is perhaps the more common one, and is used according to the present invention for decorrelation of spectral data, as this term is defined above. However, considering the fact that all decorrelation statistical methods including those listed above are related to one another, there is no intention to limit the scope of the invention to use of any specific decorrelation method. Specifically, there is no intention to limit the scope of the present invention to use of principal component analysis, as any other decorrelation statistical method may be alternatively employed. Information concerning the use and operation of the above listed decorrelation statistical methods is found in R. A. Johnson and D. W. Wichen, “Applied Multivariance Statistical Analysis”, third edition, Prentice Hall (1992) and T. W. Anderson, “An Introduction to Multivariance Statistical Analysis”, second edition, Wiley and Sons (1984), both are incorporated by reference as if fully set forth herein.

Furthermore, as will become apparent from the descriptions to follow, the implementation of a decorrelation statistical method may be done using various modifications. As the concept of the present invention is not dependent upon any specific modification, it is the intention that the scope of the present invention will not be limited to any specific modification as described below.

A brief description of the principal component analysis using a covariance matrix is given below. For further details regarding the principal component analysis, the reader is referred to Martens and Naes (1989) “Multivariate Calibration”, John Wiley & Sons, Great Britain; and to Esbensen et al., Eds. (1994) Multi Variance Analysis—in practice. Computer-aided modeling as CAMO, and the Unscrambler's User's guide, Trondheim, Norway.

Thus, the intensities of the pixels of the image at wavelength λᵢ (i=1, . . . ,N) are now considered a vector whose length is equal to the number of pixels q. Since there are N of these vectors, one for every wavelength of the measurement, these vectors can be arranged in a matrix B′ with q rows, and N columns:

$$\begin{matrix}No . of wavelengths \\ & \\ {B}^{′} = No . of pixels \begin{matrix}{B}_{11}^{′} & \cdot & \cdot & \cdot & {B}_{1 N}^{′} \\ \cdot & & & & \cdot \\ \cdot & & & & \cdot \\ \cdot & & & & \cdot \\ {B}_{q1}^{′} & \cdot & \cdot & \cdot & {B}_{qN}^{′}\end{matrix} & ( 12 )\end{matrix}$$

For each of the columns of matrix B′ defined is an average:

$${M}_{i} = \frac{1}{q} {\sum }_{i = 1}^{q} {B}_{ji}^{′} ; i = 1 … N \qquad ( 13 )$$

and a second normalized matrix B defined as:

No. of Wavelengths

$$B = No . of pixels \begin{matrix}{B}_{11}^{′} / {M}_{1} & … & {B}_{1 N}^{′} / {M}_{N} \\ ⋮ & & ⋮ \\ {B}_{q1}^{′} / {M}_{1} & … & {B}_{q N}^{′} / {M}_{N}\end{matrix} \qquad ( 14 )$$

A covariance matrix C is defined for the matrix B: C=B^{T}·B of dimensions N×N. C is diagonalized, and eigenvectors and eigenvalues related by: C·Vᵢ=μᵢ·Vᵢ where Vi are N orthogonal unit vectors and μᵢ are the eigenvalues representing the variance in the direction of the i-th unit vector Vᵢ. In general, the lowest components represent the highest variability as a function of pixels.

The products BVᵢ(i=1, . . . N) are the projections of the spectral image onto the elements of the orthogonal basis, they are vectors with q elements (q=number of pixels), and can be displayed separately as black and white images. These images may reveal features not obvious from a regular black and white image filtered at a certain wavelength or wavelength range.

The following summarizes the advantages of using spectral imaging in the detection step:

Thus, as is shown herein, the present invention enables an accurate subtraction of the background signal by identifying the exact background spectrum of the image. Other non-related spectra such as auto-fluorescence or direct scattering may also be eliminated. The background (and other non-related spectra) subtraction allows obtaining a substantially clean signal which relates solely to the actual spectral-codes, hence, the number of different responses to light that may be used are significantly increased. Additionally, as is described herein, the present invention offers an improved signal-to-noise ratio over prior art methods, and thereby increases the reliability of the classification of each object.

Therefore, the overall accuracy in the determination of the presence, absence and/or level of each of the analytes-of-interest is significantly improved by the present invention. This improvement emerges directly from the detection step in which spectral imaging is preferably used. In a spectral image, many data points in the spectrum are acquired for each picture element of the image, hence more information is available from each picture element. Moreover, the image itself is very informative by allowing, as an example, to relay on spectral data measured from picture element located at or near the center of an object rather than the edges of it.

By having the full spectrum for each picture element of the image, it is possible to use a set of responses to light and distinguish them from one another. As the objects are labeled with color components having different responses to light, it is possible to analyze the spectrum characterizing each object and to determine the exact contribution of each response. Having the full spectrum allows, in addition, eliminating any noise that does not belong to the expected responses. This can be done, for example, by measuring the spectrum at regions of the image that do not contain any object. The average spectrum in this area can serve as a reference background spectrum which is later subtracted from the spectrum of each pixel of the image.

In a preferred embodiment, it is also possible to measure a characteristic spectrum of each object and to store it in a library. In feature measurements, this library can be used for identifying the different objects. The fact that complete spectra are available, allows not only to identify the different responses to light, but also to determine the level of residual spectra in a given measurement, i e., the spectra obtained by subtracting measured spectra from corresponding archived reference spectra. The residual spectra is informative, as it can teach on the source of the noise in the system. This information can be used to improve the determination of the responses to light and it can be subtracted from the measurements if it is consistent.

Objects and Fluorochromes

The bead objects used in context of the present invention can be made, for example, of polystyrene or latex. However, other polymeric materials are acceptable including polymers selected from the following chemical groups: carbohydrate-based polymers, polyaliphatic alcohols, poly(vinyl) polymers, polyacrylic acids, polyorganic acids, polyamino acids, co-polymers, block co-polymers, tert-polymers, polyethers, naturally occurring polymers, polyimids, surfactants, polyesters, branched polymers, cyclo-polymers, polyaldehydes and mixtures thereof. Specific examples include brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphsophaze, or combinations thereof are preferable.

Representative combination polymers of which the polymeric beads are composed include for example poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio).

Most of beads which are formed from synthetic polymers such as polystyrene, polyacrylamide, polyacrylate, or latex are now commercially available from numerous sources such as Bio-Rad Laboratories (Richmond, Calif.) and LKB Produkter (Stockholm, Sweden).

Beads which are formed from natural macromolecules such as agarose, crosslinked agarose, globulin, deoxyribose nucleic acid, and liposomes are commercially available from sources such as Bio-Rad Laboratories, Pharmacia (Piscataway, N.J.), and IBF (France).

Beads which are formed from copolymers of polyacrylamide and agarose are commercially available from sources such as IBF and Pharmacia.

Surface functional groups aimed to facilitate the attachment of affinity molecules, such as antibodies or polynucleotides to the beads include, but are not limited to, carboxylates, esters, alcohols, carbamides, aldehydes, amines, sulfur oxides, nitrogen oxides, or halides.

A conventional procedure for covalently attaching an immunologically reactive species (e.g., antibody) to an object having surface carboxyl groups involves the use of a water-soluble carbodiimide. For many practical applications it is critical that the polymeric object have surface carboxyl groups available for attachment of the reactive amine- or sulfhydryl-containing compound. Such groups are preferably added to the objects by incorporating monomers containing such groups into the polymers (for example, acrylic acid, methacrylic acid, itaconic acid, and the like). Alternatively, they can be added to the objects by further chemical reaction of a polymer having other precursor reactive groups which can be converted to carboxyl groups (for example, by hydrolysis of anhydrides, such as maleic anhydride, or by oxidation of surface methylol or aldehyde end groups). Other compounds, such as diamines, dihydrazides, mercaptoalkylamines and dimercaptans can be used as linking moieties for later attachment of drugs, enzymes or other reactive species such as nanospheres. Although the preferred attaching or bonding method is by covalent linkage other methods such as adsorption can be equally used. Other novel methods such as surrounding the beads by a polymeric shell are acceptable as well.

Fluorescent fluorochromes used in this invention are of the general class known as cyanine fluorochromes, with emission wavelengths between 550 nm and 900 nm. These fluorochromes may contain methine groups and their number influences the spectral properties of the fluorochrome. The monomethine fluorochromes that are pyridines typically have blue to blue-green fluorescence emission, while quinolines have green to yellow-green fluorescence emission. The trimethine fluorochrome analogs are substantially shifted toward red wavelengths, and the pentamethine fluorochromes are shifted even further, often exhibiting infrared fluorescence emission (see, for example, U.S. Pat. No. 5,760,201).

However, it is to be understood that any other fluorochrome that is soluble in an organic solvent can be used.

In addition to fluorescent fluorochromes, related fluorochromes can be further selected from cyclobutenedione derivatives, substituted cephalosporin compounds, fluorinated squaraine compositions, symmetrical and unsymmetrical squaraines, alkylalkoxy squaraines, or squarylium compounds. Some of these fluorochromes can fluoresce at near infrared as well as at infrared wavelengths that would effectively expand the range of emission spectra up to about 1,000 nm. In addition to squaraines, i.e., derived from squaric acid, hydrophobic fluorochromes such as phthalocyanines and naphthalocyanines can be also selected as operating at longer wavelengths. Other classes of fluorochromes are equally suitable for use in context of the present invention. Some of these fluorochromes are listed herein: 3-Hydroxypyrene 5,8,10-Tri Sulfonic acid, 5-Hydroxy Tryptamine, 5-Hydroxy Tryptamine (5-HT), Acid Fuhsin, Acridine Orange, Acridine Red, Acridine Yellow, Acriflavin, AFA (Acriflavin Feulgen SITSA), Alizarin Complexon, Alizarin Red, Allophycocyanin, ACMA, Aminoactinomycin D, Aminocoumarin, Anthroyl Stearate, Aryl- or Heteroaryl-substituted Polyolefin, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, BOBO 1, Blancophor FFG Solution, Blancophor SV, Bodipy F1, BOPRO 1,Brilliant Sulphoflavin FF, Calcien Blue, Calcium Green, Calcofluor RW Solution, Calcofluor White, Calcophor White ABT Solution, Calcophor White Standard Solution, Carbocyanine, Carbostyryl, Cascade Blue, Cascade Yellow, Catecholamine, Chinacrine, Coriphosphine O, Coumarin, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH-CH3, DAPI, Diamino Phenyl Oxydiazole (DAO), Dimethylamino-5-Sulphonic acid, Dipyrrometheneboron Difluoride, Diphenyl Brilliant Flavine 7GFF, Dopamine, Eosin, Erythrosin ITC, Ethidium Bromide, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Hoechst 33258, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nile Red, Nitrobenzoxadidole, Noradrenaline, Nuclear Fast Red, Nuclear Yellow, Nylosan Brilliant Flavin E8G, Oregon Green, Oxazine, Oxazole, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Propidium Iodide, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Rose Bengal, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Texas Red, Thiazine Red R, Thioflavin S, Thioflavin TCN, Thioflavin 5, Thiolyte, Thiozol Orange, Tinopol CBS, TOTO 1, TOTO 3, True Blue, Ultralite, Uranine B, Uvitex SFC, Xylene Orange, XRITC, YO PRO 1, or combinations thereof.

Optionally such fluorochromes will contain functional groups capable of forming a stable fluorescent product with functional groups typically found in biomolecules or polymers, such as antibodies and polynucleotides, including activated esters, isothiocyanates, amines, hydrazines, halides, acids, azides, maleimides, alcohols, acrylamides, haloacetamides, phenols, thiols, acids, aldehydes and ketones.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

This example demonstrates a preparation of a sample for spectral imaging, in accordance with the present invention.

Vials containing reagents as described herein were assembled:

1. Anti-cytokine conjugated beads: a mix of 8 bead classes, each having its own color and intensity and a different antibody for a different cytokine. This vial is further referred to as vial 1.

2. Cytokine detection antibody diluted in buffer A (see below). This antibody is cross-reactive with all cytokines. This vial is further referred to below as vial 2.

3. Reporter: Streptvidin-Phycoerythrin diluted in distilled water. This vial is further referred to below as vial 3.

The following buffers were prepared:

1. Buffer A: 4×SSC.

2. Wash Buffer: 4×SSC/0.1% TWEEN 20.

In addition, a titer plate specially design for vacuum filtration through a low fluorescent membrane was used.

The reaction steps:

1. A multiple-beads stock was prepared by mixing 1 volume from vial 1 and 25 volumes from Buffer A.

2. The wells were washed with 50 μl of buffer A. The buffer was removed by vacuum.

3. 50 μl of multiple beads stock were added to each well.

4. 50 μl of analyzed samples blood cells, suspected to be infected, were added to different wells. The plate was briefly vortexed and left still for 30 minutes incubation. Thereafter, liquids were removed and the beads washed once with 50 μl of buffer A.

5. 20 μl of the detection antibody (vial 2) were added to each well. After a brief vortex the plate was left to incubate for about 30 minutes. Thereafter, liquids were removed and the beads washed once with 50 μl of buffer A.

6. 50 μl of the reporter fluorochrome (vial 3) were added to each well. After a brief vortex the plate was left to incubate for about 10 minutes. Thereafter, liquids were removed and the beads washed once with 50 μl of buffer A.

Once the above steps were completed the titer plate was ready for scanning using a spectral imaging device.

### Example 2

This example demonstrates a spectral image for multi-spectrally labeled beads. The spectral image was measured with an interferometer-based spectral imaging system. The beads were manufactured and stained by Sperotech Inc. (Libertyville Ill, USA). About 5500 beads of 5 μm in diameter were simultaneously imaged. The beads were classified into 4 different populations each population was spectrally labeled with a different fluorochrome: SKY Blue, Flash Red, Sun Coast and Nile Blue.

The spectral resolution of the measurement was a full width at half maximum (FWHM) of 15 nm at 500 nm (the FWHM varies with wavelength because with a Fourier-based spectrometer the spectral resolution is constant in the energy or wavenumber domain and it varies in the wavelength domain).

The CCD had 1280×1024 pixels, each one having an effective size of 6.7 μm×6.7 μm, and the system was used with a fore-optics that provides an effective magnification of 10 folds. The spatial resolution was therefore approximately 0.67 μm×0.67 μm for each pixel. Thus, each 5 μm bead was approximately imaged by 8×8 pixels.

The measurement time of the image was about 10 seconds.

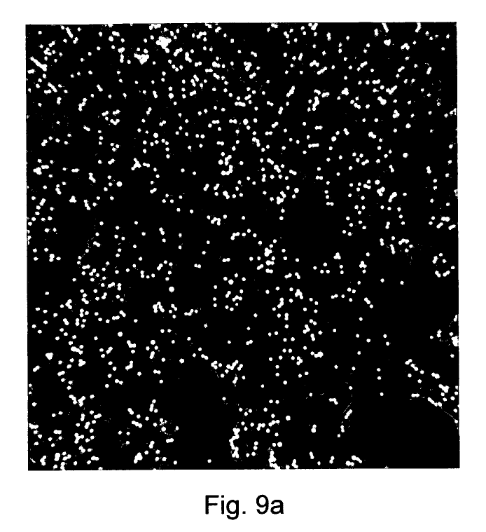

FIG. 8 shows the spectra of the different fluorochromes: SKY Blue, Flash Red, Sun Coast and Nile Blue. Evidently, these spectra are very similar and cannot be distinguished from one another using the naked eye.

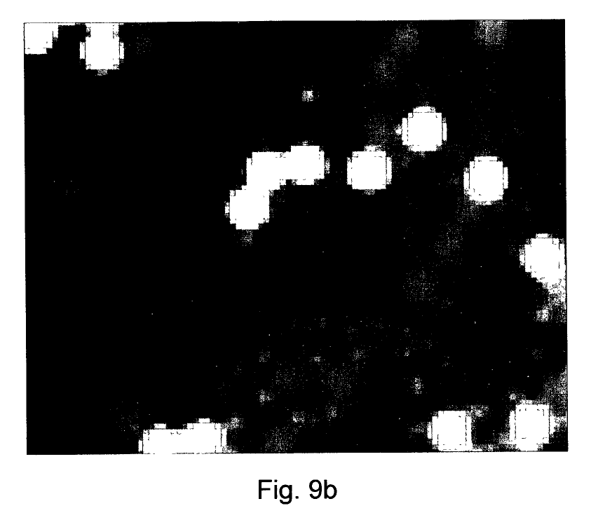

FIGS. 9a-b show the spectral image of the beads, were FIG. 9b includes the scaling in pixels showing that each bead is imaged by 8×8 pixels. Such a high spatial resolution and large field of view enable the identification of each one of the beads by using conventional image processing algorithms.

The colors shown in the image are the result of a classification algorithm, whereby each pixel having a given spectrum is colored with a predetermined artificial color. It will be appreciated that an RGB algorithm can be similarly used. Further details regarding these procedures can be found in the patent listed above.

FIGS. 9a-b therefore demonstrates the power of the invention described herein. With the adequate spectral and spatial resolution, it is possible to identify thousands of beads in a single image. By performing spectral analysis for each one of the beads, it is possible to identify the spectral-code of the bead and the level of binding that took place on its surface.

### Example 3

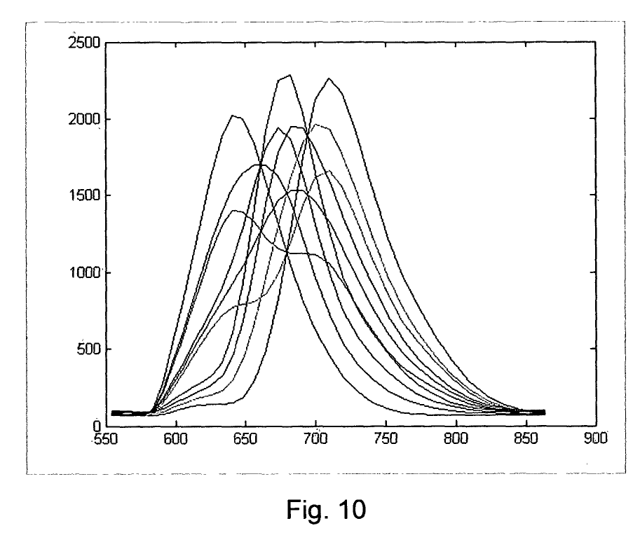

FIG. 10 shows spectra of 10 different beads which were labeled using a combinatorial labeling approach, and were analyzed using spectral imaging similar to as described under Example 2 above.

As in the previous example the spectra shown in FIG. 10 are indistinguishable to the naked eye. Although the spectra are complex, the spectral analysis of it provides a well-defined identification of each one of the spectral-coded beads.

### Example 4

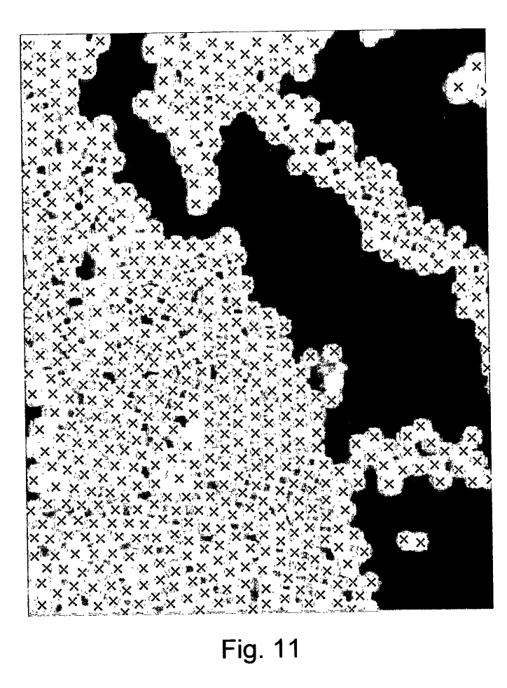

FIG. 11 shows the result of an image analysis algorithm that identifies all the beads in a spectral image. The aim of the algorithm was to detect the presence of beads in the image.

The image of the beads was measured prior to the measurement with similar conditions and stored as a reference in the computer. After a gray-scale image measurement, the normalized cross correlation between the image and the bead reference image was calculated [see, e.g., Jain, “Fundamentals of Digital Image Processing”, Prentice-Hall International Jain (1989); and J. P. Lewis, “Fast Template Matching”, Vision Interface, 120-123, (1995)]. The beads positions are identified as local maxima of the normalized cross correlation. The locations of the beads are shown as X's in FIG. 11.

Further information can be used for confirming the identification of the bead, such as testing its two dimensional intensity profile, edges and so on. As a result, such a calculation provides an accurate and reliable way for identifying the beads locations. This information is most valuable, and can be further used for calculating the average intensity of all the other parameters that are measured (such as the spectrum of a bead C_{O} and of C_{M}).

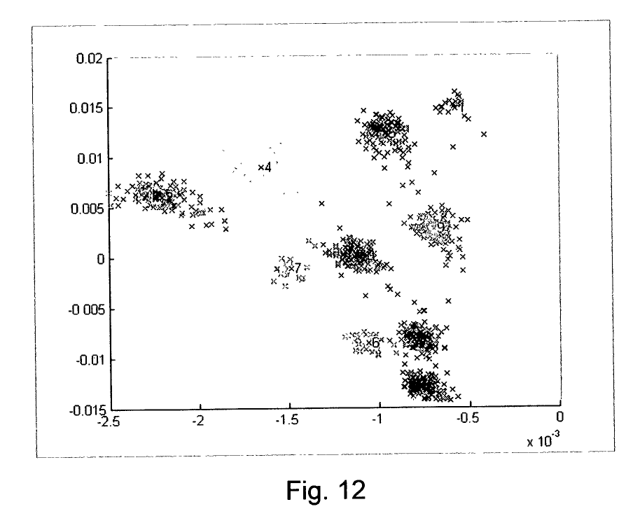

FIG. 12 shows a scatter plot of the analyzed beads spectra. The figure emphasizes the difference between the different classes of beads. It is produced by projecting the n dimensions measured spectrum of each bead on a 2-dimensional space for displaying purposes. The projection method is selected so as to maximize the distance between the different projected classes. Projection of multidimensional data onto a lower dimensional space is a known method that is used prior to classification to reduce the so-called curse of dimensionality.

The combination of fluorophores for each bead is listed hereinbelow in Table 2:

TABLE 2

| Bead | Fluorophore | Fluorophore | Fluorophore | Total |
|---|---|---|---|---|
| Type | 1 | 2 | 3 | Intensity |
| 1 | 100% | 0% | 0% | 100% |
| 2 | 0% | 100% | 0% | 100% |
| 3 | 0% | 0% | 100% | 100% |
| 4 | 33% | 67% | 0% | 100% |
| 5 | 67% | 33% | 0% | 100% |
| 6 | 0% | 33% | 67% | 100% |
| 7 | 0% | 67% | 33% | 100% |
| 8 | 33% | 0% | 67% | 100% |
| 9 | 67% | 0% | 33% | 100% |
| 10 | 33% | 33% | 33% | 100% |

### Example 5

Following is an example which demonstrates a procedure for acquisition and data processing of a sample that includes plurality of beads. The data is acquired by generating a spectral image, which, as already emphasized hereinabove, includes a plurality of intensities measured at each pixel of the image. This spectral image is then used to obtain information on the concentration or level of expression of each one of the many parameters being tested. In addition to the spectral image being measured, the procedure uses calibration data that allow translating intensity values into real concentration values.

The output for each of the plurality of beads, as will be further demonstrated, includes: (i) the number of beads for each parameter being tested; (ii) average expression intensity from each parameter being tested; (iii) standard deviation of the expression intensity from each parameter being tested; and (iv) a reliability measure.

Optionally, as further described below, the procedure may use a gray-scale image of the affinity moiety. This information is available in embodiments in which there are no cross-talks between the objects colors and the affinity moiety color(s).

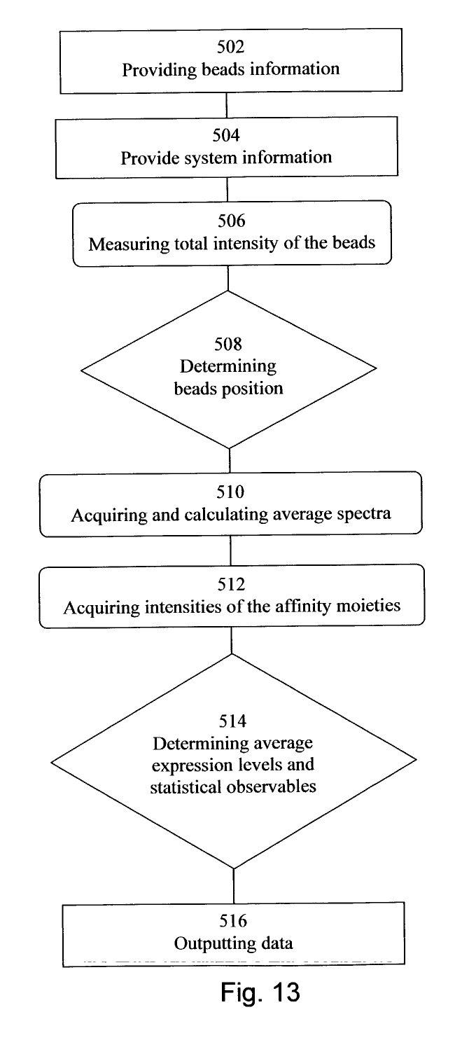

Reference is now made to FIG. 13, which is a simplified flowchart of the procedure. Hence, in a first step, designated by Block 502, beads information is provided. The beads information includes: (i) number of bead classes; (ii) beads size and shape; (iii) typical background and autofluorescence values and beads light scattering values; and (iv) fluorescence spectra of each one of the bead classes.

In a second step, designated by Block 504, system parameters are provided. The system parameters include (i) X,Y offset and step size which needed to scan the sample; (ii) calibration parameters for correct spectral measurement; (iii) focusing calibration and mechanical/optical setup parameters; and (iv) sensor offset, exposure and other acquisition parameters.

In a third step of the procedure, designated by Block 506, the total intensity of the beads is measured. This step is includes the following substeps: (i) activating the excitation light; (ii) optimizing the focus on beads, this can be done either manually or automatically; and (iii) acquiring a gray-level image measuring the spectrally integrated intensity of the beads. The gray-level image is referred to hereinafter as “Segmentation image”.

In a fourth step, designated by Block 508, the beads location in the image is determined automatically by imposing intensity threshold on the image. As the beads' intensities are considerably stronger than background level, the location of each bead is determined to a high accuracy, and each bead is attributed to a well defined number of pixels in the image. Each pixel in the image, other than a pixel being attributed to a bead, is automatically defined as a background pixel. In addition, in this step, the beads shapes and sizes are also determined so as to filter out signals from other objects.

In a fifth step of the procedure, designated by Block 510, the average spectrum of each bead is acquired and calculated. This step is done by obtaining a spectral image and extracting the spectrum of each of the beads that were detected in the Segmentation image. The spectrum of a particular bead may be defined in more than one way. For example, by calculating an average spectrum over all the pixels imaging the particular bead. The information on the exact pixels that should be averaged for each bead is provided by the fourth step as detailed above with reference to Block 508. Other known algorithms may also be used for calculating the spectra of the beads. In any case, each bead is uniquely characterized by its normalized spectrum, irrespectively of the algorithm used for calculating it. Using the beads information as provided in the first step, each bead is classified as one of the plurality of beads classes. Various classification schemes can be used. In this example a Minimal Square Error (MSE) criteria is used, matching the spectra of an unknown bead to each of the library spectra. Bead class is defined as the class for which the MSE was minimal.

Once the fifth step is completed the excitation light is changed so as to match the signal emitted from the affinity moieties. Block 512 represents a sixth step of the procedure in which the intensities of the affinity moieties are acquired. A detailed description of the sixth step is now provided.

Hence, in the sixth step a second gray-level image is acquired using the excitation light matching the affinity moieties. As stated hereinabove, the signals from the affinity moieties are directly related to analyte which occupy the beads. Hence, the second gray-level image measures the expression level of the analyte-of-interest. From the second gray-level image, an average background value is calculated. Then, for each bead, an intensity value is calculated, for example by averaging as further detailed hereinabove with respect to the spectral image.

In a seventh step of the procedure, designated by Block 514, average expression levels are determined, and statistical observables for the various expression levels are calculated. Hence, using the calibration parameters and the intensities values of the beads an offset level of each bead is subtracted. All resultant values are then categorized according the classes which were extracted in the fifth step of the procedure. For each beads class, a plurality of statistical observables (e.g., median, average, standard deviation) is calculated.

In an eighth step, designated by Block 516, the final result are calculated and outputted to an external device (memory media, display, printer and the like). For each of the plurality of analytes, the final results are calculated according to the specific requirements of the assay. For example, subtracting the background from the analyte signal, or subtracting measured values known as negative control from the unknown sample values.

The above procedure may also be supplemented by an additional step of reducing scattering effects by measuring, the spectrum that is scattered from one bead to its neighbors, thereby providing, for each bead, a scattering profile. The scattering profile is then subtracted from the image by using de-convolution algorithms. For example, if it is found that a red-colored bead increases the red fluorescence of its neighbors in an intensity that is equal to 10% of its own intensity, the red spectrum from all the neighbors of the red beads is reduced by 10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of detecting the presence, absence and/or level of a plurality of analytes-of-interest in a sample, the method comprising:
(a) providing a plurality of objects, each of said plurality of objects having a predetermined, measurable and different imagery characteristic, and further having a predetermined and specific affinity to one analyte of the plurality of analytes-of-interest, each said imagery characteristic corresponding to one said predetermined specific affinity, hence each said imagery characteristic corresponds to one analyte of the plurality of analytes-of interest;
(b) providing at least one affinity moiety having a predetermined and specific affinity or predetermined and specific affinities to the plurality of analytes-of-interest, each said affinity moiety having a predetermined, measurable response to light;
(c) combining said objects, said at least one affinity moiety and the sample under conditions for affinity binding; and
(d) simultaneously determining, for each object of said plurality of objects an imagery characteristic, and for at least a portion of said at least one affinity moiety a response to light, thereby detecting the presence, absence and/or level of the plurality of analytes-of-interest in the sample.

2. The method of claim 1, wherein said predetermined, measurable and different imagery characteristic is selected from the group consisting of a unique size, a unique geometrical shape and a unique response to light.

3. The method of claim 2, wherein said step (d) is by a spectral imaging device operable to construct a spectral image of the sample.

4. The method of claim 3, wherein said spectral image comprises at least two colors.

5. The method of claim 3, wherein said spectral image comprises at least three colors.

6. The method of claim 3, wherein said spectral image comprises at least four colors.

7. The method of claim 2, wherein said step (d) comprises determining, for each object, a wavelength value and an intensity value.

8. The method of claim 7, wherein said wavelength value is used to determine a presence of a particular analyte of said plurality of analytes-of-interest in the sample.

9. The method of claim 7, wherein said intensity value is used to determine a level of a particular analyte of said plurality of analytes-of-interest in the sample.

10. The method of claim 1, wherein the analytes-of-interest are dissolved, suspended or emulsed in a solution.

11. The method of claim 1, wherein the analytes-of-interest are selected from the group consisting of antigens, antibodies, receptors, haptens, enzymes, proteins, peptides, nucleic acids, drugs, hormones, chemicals, polymers, pathogens, toxins, and combination thereof.

12. The method of claim 1, wherein the analytes-of-interest are selected from the group consisting of viruses, bacteria, cells and combination thereof.

13. The method of claim 2, wherein said unique geometrical shape is selected from the group consisting of a spherical shape, a pyramidal shape, a flat shape and an irregular shape.

14. The method of claim 1, wherein a portion of said plurality of objects are beads.

15. The method of claim 1, wherein a portion of said plurality of objects are disks.

16. The method of claim 1, wherein said plurality of objects are predetermined spatial x-y locations on two-dimensional array.

17. The method of claim 16, wherein said two-dimensional array is a micro-array chip.

18. The method of claim 1, wherein said objects are of micrometer size.

19. The method of claim 1, wherein each of said plurality of objects comprises a predetermined combination of color-components, each color-component is selected from the group consisting of fluorochromes, chromogenes, quantum dots, nanocrystals, nanoprisms, nanobarcodes, scattering metallic objects, resonance light scattering objects and solid prisms.

20. The method of claim 19, wherein each of said color-components is characterized by a predetermined concentration level.

21. The method of claim 19, wherein each-of said fluorochromes is selected from the group consisting of Aqua, Texas-Red, FITC, rhodamine, rhodamine derivative, fluorescein, fluorescein derivative, cascade blue, Cyanine and Cyanine derivatives.

22. The method of claim 1, wherein said specific affinity of each of said plurality of objects and said specific affinity of each of said at least one affinity moiety are independently capable of binding to an analyte by means of an ionic linkage or a non-ionic linkage.

23. The method of claim 1, wherein said specific affinity of each of said plurality of objects and said specific affinity of each of said at least one affinity moiety are independently capable of binding to an analyte by means of covalent linkage or a non-covalent linkage.

24. The method of claim 1, wherein said specific affinity of each object of said plurality of objects is adsorbed onto a surface of said object.

25. The method of claim 1, wherein said specific affinity of each object of said plurality of objects is covalently linked to said object.

26. The method of claim 1, wherein said specific affinity of each of said plurality of objects and said specific affinity of each of said at least one affinity moiety are independently selected from the group consisting of a nucleic acid, an antibody, an antigen, a receptor, a ligand, an enzyme, a substrate and an inhibitor.

27. The method of claim 1, further comprising repeating said step (c) a plurality of times, each time on a different x-y location of a two-dimensional platform.

28. The method of claim 27, wherein said two-dimensional platform is a microtiter plate.

29. The method of claim 27, wherein said step (d) is performed for each x-y location separately.

30. The method of claim 27, wherein said step (d) is performed simultaneously for all x-y locations.

31. The method of claim 1, further comprising repeating said step (d) at least once, so as to optimize a signal-to-noise ratio.

32. The method of claim 3, further comprising performing at least one calibration spectral imaging measurement prior to said step (d).

33. The method of claim 2, wherein responses to light of said plurality of objects and responses to light of said at least one moiety are determined simultaneously.

34. The method of claim 2, wherein responses to light of said plurality of objects and responses to light of said at least one moiety are determined separately and independently.

35. The method of claim 1, wherein responses to light of said at least one moiety are determined by gray-level imaging.

36. The method of claim 3, further comprising subtracting background spectra from said spectral image, said background spectra are collected from a regions of said image which are characterized by absence of objects.

37. The method of claim 3, further comprising magnifying said spectral image by a magnification factor, said magnification factor is from 1 to 100.

38. The method of claim 2, further comprising selecting an optimal excitation and emission spectrum of each of said plurality of objects.

39. The method of claim 38, wherein said selecting an optimal excitation and emission spectrum is by an epi-fluorescent setup which comprises at least one spectral filter.

40. The method of claim 1, wherein said step (d) is effected by a procedure selected from a group consisting of a principle component analysis, a principle component regression and a spectral decomposition.

41. The method of claim 2, wherein said step (d) comprises using a library of reference spectra characterizing said plurality of objects.

42. The method of claim 3, wherein said spectral imaging device comprises a dispersion element and a detector.

43. The method of claim 42, wherein said dispersion element is an interferometer.

44. The method of claim 43, wherein said interferometer is selected from the group consisting of a moving type interferometer, a Michelson type interferometer and a Sagnac type interferometer.

45. The method of claim 42, wherein said dispersion element is at least one filter, selected so as to collect spectral data of intensity peaks characterizing a response to light of each of said plurality of objects.

46. The method of claim 45, wherein each of said at least one filter is independently selected from the group consisting of an acousto-optic tunable filter and a liquid-crystal tunable filter.

47. The method of claim 42, wherein said dispersion element is selected from the group consisting of a grating and a prism.

48. The method of claim 42, wherein said detector is selected from the group consisting of a CCD detector, a C-MOS detector, a line-scan array, an array of photo diodes and a photomultiplier.

49. The method of claim 42, wherein said spectral imaging device further comprises at least one light source.

50. The method of claim 49, wherein said at least one light source is selected from the group consisting of Mercury lamp, Xenon lamp, Tungsten lamp, Halogen lamp, laser light source, Metal-Halide lamp.

51. The method of claim 3, wherein said step (d) comprises:
(i) illuminating the sample with incident light; and
(ii) collecting exiting light from the sample so as to acquire a spectrum of each object of said plurality of objects.

52. The method of claim 51, wherein said exiting light is reflected from the sample.

53. The method of claim 51, wherein said exiting light is transmitted through the sample.

54. The method of claim 51, wherein said exiting light is emitted from the sample.

55. The method of claim 51, further comprising positioning at least a portion of said plurality of objects on a two-dimensional platform, prior to said step (i).

56. The method of claim 51, wherein said positioning is effected by a procedure selected from the group consisting of printing and gluing.

57. The method of claim 55, wherein said two-dimensional platform is a microtiter plate.

58. The method of claim 55, wherein said two-dimensional platform is a microscope slide.

59. The method of claim 51, further comprising using at least one filter to adjust a spectrum of said incident light.

60. The method of claim 51, further comprising substantially filtering out an exciting wavelength of said incident light while collecting said exiting light.

61. The method of claim 60, wherein said filtering out exciting wavelength is by an optical device selected from the group consisting of a dichroic mirror, a dark-field objective lens, a phase contrast device and a Numarski-prism.

62. The method of claim 51, further comprising acquiring an intensity value of each picture element of said at least a portion of the sample.

63. The method of claim 62, wherein said intensity value is used to determine a level of a particular analyte of said plurality of analytes-of-interest in the sample.

64. The method of claim 51, wherein said step (ii) is characterized by spectral resolution ranging between 1 nm and 50 nm and spatial resolution ranging between 0.1 mm and 1.0 mm.

65. The method of claim 51, further comprising generating individual spectra-images from spectra acquired in said step (ii).

66. The method of claim 42, wherein said illuminating is by at least one light source selected from the group consisting of Mercury lamp, Xenon lamp, Tungsten lamp, Halogen lamp, laser light source, Metal-Halide lamp.

67. The method of claim 3, wherein said spectral imaging device comprises an interferometer and a detector, said interferometer comprising two mirrors and one beam-splitter, and said detector comprising a two dimensional array of detector elements.

68. The method of claim 67, wherein said detector is a CCD detector.

69. The method of claim 67, wherein said step (d) comprises:
(i) collecting incident light simultaneously from said plurality of objects;
(ii) passing said incident light through said interferometer, so that said light is first split into two coherent beams having an optical path difference therebetween, and then said two coherent beams recombine to interfere with each other to form an exiting light;
(iii) focusing said exiting light on said detector, so that each of said detector elements produces a signal which is a particular linear combination of light intensity emitted by a respective object of said plurality of objects, said linear combination is a function of said optical path difference;
(iv) simultaneously scanning said optical path difference for said plurality of objects; and
(v) recording said signals of each of said detector elements as function of time.

70. The method of claim 69, further comprising passing said incident light through a collimator, prior said step (ii), said collimator designed and configured such that said light is simultaneously collected and collimated for each of said plurality of objects.

71. The method of claim 69, wherein said collimator is an afocal telescope.

72. The method of claim 69, wherein said collimator is a microscope.

73. The method of claim 69, wherein said simultaneously scanning said optical path difference is by rigidly rotating said beam-splitter and said two mirrors around an axis perpendicular to a plane formed by said two coherent beams.

74. The method of claim 69, wherein said interferometer further comprises a first periscope mirror, a second periscope mirror and a double sided mirror having a first side and a second side, and further wherein said simultaneously scanning said optical path difference is by rotating said double sided mirror around an axis perpendicular to a plane formed by said two coherent beams, in a manner that said incident light:
encounters said first side of said double sided mirror,
encounters said first periscope mirror,
splits and recombined in said beam-splitter and said two mirrors;
encounters said second periscope mirror, and
encounters said second side of said double sided mirror.

75. The method of claim 69, wherein said interferometer further comprises a single large mirror, and further wherein said simultaneously scanning said optical path difference is by rotating said large mirror around an axis perpendicular to a plane formed by said two coherent beams, in a manner that said incident light:
encounters said large mirror;
splits and recombined in said beam-splitter and said two mirrors; and
reflected by said large mirror.

76. The method of claim 69, wherein said beam-splitter and said two mirrors are combined in a single rigid element, shaped as a prism.

77. The method of claim 69, wherein said beam-splitter and said two mirrors are combined in a single rigid element, shaped as a grating.

78. The method of claim 69, wherein said beam-splitter and said two mirrors are combined in a single rigid element, shaped as a combination of a prism and a grating.

79. The method of claim 69, further comprising simultaneously transferring all data in real time from all said elements of said detector array to a computer, and displaying an image on an output device.

80. The method of claim 79, wherein said output device is a screen.

81. The method of claim 79, wherein said output device is a printed image.

82. A system for detecting the presence, absence and/or level of a plurality of analytes-of-interest in a sample, the system comprising:
(a) a plurality of objects, each of said plurality of objects having a predetermined, measurable and different imagery characteristic, and further having a predetermined and specific affinity to one analyte of the plurality of analytes-of-interest, each said predetermined imagery characteristic corresponding to one said predetermined specific affinity, hence each said imagery characteristic corresponds to one analyte of the plurality of analytes-of interest;
(b) at least one affinity moiety having a predetermined and specific affinity or predetermined and specific affinities to the plurality of analytes-of-interest, each said affinity moiety having a predetermined, measurable response to light;
(c) a container for combining said objects, said at least one affinity moiety and the sample under conditions for affinity binding; and
(d) a determinator for simultaneously determining, for each object of said plurality of objects an imagery characteristic, and for at least a portion of said at least one affinity moiety a response to light, thereby detecting the presence, absence and/or level of the plurality of analytes-of-interest in the sample.

83. The system of claim 82, wherein said predetermined, measurable and different imagery characteristic is selected from the group consisting of a unique size, a unique geometrical shape and a unique response to light.

84. The system of claim 83, wherein said unique geometrical shape is selected from the group consisting of a spherical shape, a pyramidal shape, a flat shape and an irregular shape.

85. The system of claim 83, wherein said determinator is a spectral imaging device operable to construct a spectral image of the sample.

86. The system of claim 85, wherein said spectral image comprises at least two colors.

87. The system of claim 85, wherein said spectral image comprises at least three colors.

88. The system of claim 85, wherein said spectral image comprises at least four colors.

89. The system of claim 83, wherein said determinator is operable to determine, for each object, a wavelength value and an intensity value.

90. The system of claim 89, wherein said determinator is operable to determine a presence of a particular analyte of said plurality of analytes-of-interest in the sample, based on said wavelength value.

91. The system of claim 89, wherein said determinator is operable to determine a level of a particular analyte of said plurality of analytes-of-interest in the sample, based on said intensity value.

92. The system of claim 82, wherein the analytes-of-interest are dissolved, suspended or emulsed in a solution.

93. The system of claim 82, wherein the analytes-of-interest are selected from the group consisting of antigens, antibodies, receptors, haptens, enzymes, proteins, peptides, nucleic acids, drugs, hormones, chemicals, polymers, pathogens, toxins, and combination thereof.

94. The system of claim 82, wherein the analytes-of-interest are selected from the group consisting of viruses, bacteria, cells and combination thereof.

95. The system of claim 83, wherein said unique geometrical shape is selected from the group consisting of a spherical shape, a pyramidal shape, a flat shape and an irregular shape.

96. The system of claim 82, wherein a portion of said plurality of objects are beads.

97. The system of claim 82, wherein a portion of said plurality of objects are disks.

98. The system of claim 82, wherein said plurality of objects are predetermined spatial x-y locations on two-dimensional array.

99. The system of claim 98, wherein said two-dimensional array is a micro-array chip.

100. The system of claim 82, wherein said objects are of micrometer size.

101. The system of claim 83, wherein each of said plurality of objects comprises a predetermined combination of color-components, each color-component is selected from the group consisting of fluorochromes, chromogenes, quantum dots, nanocrystals, nanoprisms, nanobarcodes, scattering metallic objects, resonance light scattering objects and solid prisms.

102. The system of claim 101, wherein each of said color-components is characterized by a predetermined concentration level.

103. The system of claim 101, wherein each of said fluorochromes is selected from the group consisting of Aqua, Texas-Red, FITC, rhodamine, rhodamine derivative, fluorescein, fluorescein derivative, cascade blue, Cyanine and Cyanine derivatives.

104. The system of claim 82, wherein said specific affinity of each of said plurality of objects and said specific affinity of each of said at least one affinity moiety are independently capable of binding to an analyte by means of an ionic linkage or a non-ionic linkage.

105. The system of claim 82, wherein said specific affinity of each of said plurality of objects and said specific affinity of each of said at least one affinity moiety are independently capable of binding to an analyte by means of covalent linkage or a non-covalent linkage.

106. The system of claim 82, wherein said specific affinity of each object of said plurality of objects is adsorbed onto a surface of said object.

107. The system of claim 82, wherein said specific affinity of each object of said plurality of objects is covalently linked to said object.

108. The system of claim 82, wherein said specific affinity of each of said plurality of objects and said specific affinity of each of said at least one affinity moiety are independently selected from the group consisting of a nucleic acid, an antibody, an antigen, a receptor, a ligand, an enzyme, a substrate and an inhibitor.

109. The system of claim 82, wherein said container comprises a plurality of x-y location on a two-dimensional platform.

110. The system of claim 109, wherein said two-dimensional platform is a microtiter plate.

111. The system of claim 109, wherein said determinator is operable to process each x-y location separately.

112. The system of claim 109, wherein said determinator is operable to process all x-y locations simultaneously.

113. The system of claim 83, wherein said determinator is operable to simultaneously determine responses to light of said plurality of objects and responses to light of said at least one moiety.

114. The system of claim 83, wherein said determinator is operable to simultaneously determine responses to light of said plurality of objects and responses to light of said at least one moiety one at a time.

115. The system of claim 82, wherein said determinator is operable to generate a gray-level image of responses to light of said at least one moiety.

116. The system of claim 85, further comprising a background subtractor for collecting and subtracting background spectra from said spectral image, said background spectra are collected from a regions of said image which are characterized by absence of objects.

117. The system of claim 85, further comprising a magnifier for magnifying said spectral image by a magnification factor, said magnification factor is from 1 to 100.

118. The system of claim 83, further comprising an epi-fluorescent setup which comprises at least one filter for selecting an optimal excitation and emission spectrum of each of said plurality of objects.

119. The system of claim 83, wherein said determinator comprises a spectral analyzer operable to perform a procedure selected from a group consisting of a principle component analysis, a principle component regression and a spectral decomposition.

120. The system of claim 83, wherein said determinator communicates with a library of reference spectra characterizing said plurality of objects.

121. The system of claim 85, wherein said spectral imaging device comprises a dispersion element and a detector.

122. The system of claim 121, wherein said dispersion element is an interferometer.

123. The system of claim 122, wherein said interferometer is selected from the group consisting of a moving type interferometer, a Michelson type interferometer and a Sagnac type interferometer.

124. The system of claim 121, wherein said dispersion element is at least one filter, selected so as to collect spectral data of intensity peaks characterizing a response to light of each of said plurality of objects.

125. The system of claim 124, wherein each of said at least one filter is independently selected from the group consisting of an acousto-optic tunable filter and a liquid-crystal tunable filter.

126. The system of claim 121, wherein said dispersion element is selected from the group consisting of a grating and a prism.

127. The system of claim 121, wherein said detector is selected from the group consisting of a CCD detector, a C-MOS detector, a line-scan array, an array of photo diode array and a photomultiplier.

128. The system of claim 121, wherein said spectral imaging device further comprises at least one light source.

129. The system of claim 128, wherein said at least one light source is selected from the group consisting of Mercury lamp, Xenon lamp, Tungsten lamp, Halogen lamp, laser light source, Metal-Halide lamp.

130. The system of claim 83, wherein said determinator comprises:
(i) at least one light source for illuminating the sample with incident light ; and
(ii) a collector for collecting exiting light from the sample so as to acquire a spectrum of each object of said plurality of objects.

131. The system of claim 130, wherein said exiting light is reflected from the sample.

132. The system of claim 130, wherein said exiting light is transmitted through the sample.

133. The system of claim 130, wherein said exiting light is emitted from the sample.

134. The system of claim 130, further comprising at least one filter for adjusting a spectrum of said incident light.

135. The system of claim 130, further comprising an optical device for substantially filtering out an exciting wavelength of said incident light while collecting said exiting light.

136. The system of claim 135, wherein said optical device is selected from the group consisting of a filter, a dichroic mirror, a dark-field objective lens, a phase contrast device and a Numarski-prism.

137. The system of claim 130, wherein said collector is characterized by spectral resolution ranging between 1 nm and 50 nm and spatial resolution ranging between 0.1 mm and 1.0 mm.

138. The system of claim 130, wherein said spectral imaging device is operable to generate individual spectra-images from spectra acquired by said collector.

139. The system of claim 121, wherein said at least one light source is selected from the group consisting of Mercury lamp, Xenon lamp, Tungsten lamp, Halogen lamp, laser light source, Metal-Halide lamp.

140. The system of claim 85, wherein said spectral imaging device comprises an interferometer and a detector, said interferometer comprising two mirrors and one beam-splitter, and said detector comprising a two dimensional array of detector elements.

141. The system of claim 140, wherein said detector is a CCD detector.

142. The system of claim 140, further comprising a collimator designed and configured such that light is simultaneously collected and collimated for each of said plurality of objects.

143. The system of claim 140, wherein said collimator is an afocal telescope.

144. The system of claim 140, wherein said collimator is a microscope.

145. The system of claim 140, wherein said beam-splitter and said two mirrors are operable to rotate rigidly about a predetermined axis.

146. The system of claim 140, wherein said interferometer further comprises a first periscope mirror, a second periscope mirror and a double sided mirror having a first side and a second side, and further wherein said double sided mirror is operable to rotate about a predetermined axis.

147. The system of claim 140, wherein said interferometer further comprises a single large mirror, operable to rotate about a predetermined axis.

148. The system of claim 140, wherein said beam-splitter and said two mirrors are combined in a single rigid element, shaped as a prism.

149. The system of claim 140, wherein said beam-splitter and said two mirrors are combined in a single rigid element, shaped as a grating.

150. The system of claim 140, wherein said beam-splitter and said two mirrors are combined in a single rigid element, shaped as a combination of a prism and a grating.

151. The system of claim 140, further comprising a transmitting unit for simultaneously transferring all data in real time from all said elements of said detector array to a computer, and displaying an image on an output device.

152. The system of claim 151, wherein said output device is a screen.

153. The system of claim 151 , wherein said output device is a printed image.
